(19) United States
(12) Patent Application Publication
(10) Pub. No.: US 2026/0027169 A1
(43) Pub. Date: Jan. 29, 2026

(54) CURATING A BIOFILM USING BIOENGINEERED BACTERIA

(71) Applicant: AHP Health Innovation LLC, Elm Grove, WI (US)

(72) Inventors: Benacer Douadi, Elm Grove, WI (US); Maxwell B. Meinerz, Elm Grove, WI (US)

(21) Appl. No.: 19/282,342

(22) Filed: Jul. 28, 2025

Related U.S. Application Data
(60) Provisional application No. 63/676,737, filed on Jul. 29, 2024.

(51) Int. Cl. A61K 35/747; A61K 8/21; A61K 8/60; A61K 8/99; A61K 33/24; A61K 47/26; A61P 1/02; A61Q 11/00; C12N 1/20; C12N 15/74; C12R 1/225; C12R 1/46

(52) U.S. Cl. CPC A61K 35/747; A61K 8/21; A61K 8/60; A61K 8/99; A61K 33/24; A61K 47/26; A61P 1/02; A61Q 11/00; C12N 1/20; C12N 15/746; C12R 2001/225; C12R 2001/46

(57) ABSTRACT

Methods and compositions for treating an oral cavity of a subject to reduce or prevent instances of dental caries at one or more substrates at the oral cavity are disclosed herein. A bacterium capable of producing a biofilm is administered into the oral cavity of the subject, and, following the administration of the composition comprising the bacterium capable of producing the biofilm into the oral cavity, a pH level, at the one or more substrates at the oral cavity, is increased or stabilized to a pH level at or above 5.5.

### RELATED APPLICATION

This disclosure claims priority to U.S. provisional patent application No. 63/676,737, filed on Jul. 29, 2024, the disclosure of which is hereby incorporated by reference.

### SEQUENCE LISTING XML

The instant application contains sequence listings, which have been submitted in XML file format by electronic submission and are hereby incorporated by reference in their entirety. The XML file, created on Jul. 25, 2025, is named 85024.1.USU1 Sequence Listing.xml, and is 14,242 bytes in size.

### TECHNICAL FIELD

This disclosure relates generally to compositions and methods for bioengineered bacteria. Such embodiments disclosed herein can bioengineer one or more bacterium to thereby adapt such bacterium to better adhere to a substrate. An exemplary application is disclosed herein relating to the use of one or more bioengineered bacterium to curate a biofilm at one or more substrates at an oral cavity to thereby help to reduce or prevent instance of oral disease, such as to reduce or prevent instances of dental caries at the one or more substrates at the oral cavity.

### BACKGROUND

Streptococcus mutans is a bacteria known to be the main etiological agent in the formation of dental caries and promotes tooth decay through the ability to adhere to surfaces and produce large amounts of acid. Dental caries, the disease that creates dental cavities, causes over $100 Billion of damage to the mouths of human beings each year as a transmissible and preventable disease process.

Currently traditional products like toothpaste, floss, oral rinses, and chewing gum are designed and used with the end goal of disinfecting the mouth and removing bacteria from the surfaces of the teeth and gums. The thin layer of bacteria and other organic materials that begins to form on a surface of a tooth almost immediately after it has been cleaned is called a biofilm. As that biofilm matures it begins to create acid that damages teeth and causes cavities. By removing the biofilm, the oral cavity can remain healthy, however, adequate technique and consistency of dental hygiene practices (e.g., brushing, flossing, rinsing, etc.) are required to keep a healthy mouth. Typical oral hygiene practices include using conventional products (e.g., toothpaste, floss, mouth rinse, gum, etc.). These conventional products are designed to be antimicrobial and antibacterial with the objective of destroying the natural oral biofilm state by killing all bacteria and microbes naturally present at, for instance, the enamel, exposed dentin, cementum, and gingival tissue. However, these typical oral hygiene practices and guidelines tend to provide a short-lived state of moderate sterility and thus can be inadequate long term in reducing or preventing instances of dental caries.

### SUMMARY

Embodiments are disclosed herein relating to bioengineered bacteria. Such embodiments disclosed herein can bioengineer one or more bacterium to thereby adapt such bacterium to better adhere to a substrate. As a result, embodiments disclosed herein can leverage such improved adherence of one or more bioengineered bacteria to achieve one or more desired results.

An exemplary application is disclosed herein relating to leveraging such bioengineered bacteria to reduce or prevent instances of oral disease. Such exemplary application can leverage such bioengineered bacteria to deliver one or more oral health therapeutic effects. For instance, one or more bioengineered bacterium can be delivered to an oral cavity of a subject, where the one or more bioengineered bacterium is adapted to better adhere to an oral cavity substate (e.g., one or more teeth, gingival tissue, etc.). Depending on the characteristics of the one or more improved adherence bioengineered bacterium, the bioengineered bacterium can deliver one or more oral therapeutic effects while adhered to the oral cavity substrate. For instance, some applications can deliver one or more improved adherence bioengineered bacterium having one or more oral health therapeutic characteristics (e.g., hydrogen peroxide production when in the presence of the oral cavity) to the oral cavity substrate to deliver the one or more oral health therapeutic characteristics to the oral cavity substrate while the one or more improved adherence bioengineered bacterium are adhered to the oral cavity substrate. In additional or alternative instances, some applications can deliver one or more benign, improved adherence bioengineered bacterium to the oral cavity substrate and an oral therapeutic effect can result from the ability of the delivered one or more benign, improved adherence bioengineered bacterium to occupy space at the oral substate and thus “crowd out” malignant bacterium that could otherwise adhere to the oral cavity substrate in the absence of the delivered one or more benign, improved adherence bioengineered bacterium to the oral cavity substrate.

The inventors have observed that dental caries is a disease process caused when a thin layer of bacteria on the surface of a tooth, called a biofilm, falls out of homeostasis and into an acid generating environment dominated by bacteria who thrive in an acidic environment. Oral microbes which create acid as a metabolic byproduct, and which thrive in an acidic environment are known as pathogenic. The inventors have observed that as the biofilm matures and becomes more pathogenic, the pH drops inside of the biofilm, which resides on the surface of the tooth. A pH of 5.5 or less at the surface of the tooth begins to cause that tooth to lose mineral content and cause a lesion on the tooth, which is commonly called a dental cavity. These lesions are responsible for the billions of dollars per year spent on dental restorative services like fillings, crowns, and root canals. Thus, the inventors have discovered that curating a biofilm at the oral cavity using one or more bioengineered bacterium can help to discourage or prevent formation of an acid generating environment at that the oral cavity substrate where the one or more bioengineered bacterium are adhered to the oral cavity substrate. The inventors have demonstrated with the data disclosed herein that adherence of such one or more bioengineered bacterium to the oral cavity substrate can act to curate biofilm formation at the oral cavity substrate in a manner that can discourage or prevent formation of a malignant acid generating environment at that the oral cavity substrate, which can act to provide more long term oral hygiene benefit than traditional antimicrobial and antibacterial oral products (e.g., traditional toothpaste and mouthwash).

One embodiment disclosed herein includes a method of treating an oral cavity of a subject to reduce or prevent instances of dental caries at one or more substrates at the oral cavity. This method embodiment includes the steps of: administering into the oral cavity of the subject a composition comprising a bacterium capable of producing a biofilm; and, after administering into the oral cavity the composition comprising the bacterium capable of producing the biofilm, increasing or stabilizing a pH level, at the one or more substrates at the oral cavity, above 5.5. For instance, the method could include administering into the oral cavity of the subject a composition comprising a bacterium capable of producing a biofilm; and, after administering into the oral cavity the composition comprising the bacterium capable of producing the biofilm, increasing a pH level, at the one or more substrates at the oral cavity, to a pH range from 6.0 to 7.5.

In a further embodiment of this method, the selected bacterium is Streptococcus oligofermentans. This bacterium (e.g., Streptococcus oligofermentans) is bioengineered to include a cell surface protein antigen I/II, spaP gene. Thus, this bacterium can produce hydrogen peroxide, thereby increasing the pH level.

In a further embodiment of this method, the bacterium capable of producing the biofilm can be bioengineered to express a surface binding protein gene. For example, the method can further include adhering the composition, comprising the bacterium capable of producing the biofilm and bioengineered to include the surface binding protein gene, to the one or more substrates at the oral cavity at least via the surface binding protein gene, and the pH level, at the one or more substrates at the oral cavity, can be increased or stabilized above 5.5 after the composition is so adhered to the one or more substrates at the oral cavity. For instance, the one or more substrates at the oral cavity can include one or more teeth, and the surface binding protein gene of the bioengineered bacterium of the composition can adhere at least to the one or more teeth at the oral cavity and the pH level, at the one or more teeth, can be increased or stabilized above 5.5 after the surface binding protein gene is adhered to the one or more teeth at the oral cavity. In some such applications, after adhering the composition comprising the bacterium capable of producing the biofilm and bioengineered to include the surface binding protein gene to the one or more teeth at the oral cavity at least via the surface binding protein gene, the method can include reducing or preventing the growth of a pathogenic bacteria at the one or more teeth at the oral cavity to which the composition is adhered. This could include, when the composition comprising the bacterium capable of producing the biofilm and bioengineered to include the surface binding protein gene is adhered to the one or more teeth at the oral cavity, the bacterium producing hydrogen peroxide at the one or more teeth to which it is adhered to thereby increase or stabilize the pH level at the one or more teeth above 5.5.

In a further embodiment of this method, the bacterium can be Lactobacillus reuteri. This bacterium can be bioengineered to include a mucus-adhesion promoting protein. In some further such examples, the composition can further include at least one of: sucrose, glucose, maltose, or xylitol. As one particular such example, the composition can include the Lactobacillus reuteri bacterium and xylitol, and the Lactobacillus reuteri bacterium can be bioengineered to include a mucus-adhesion promoting protein.

In a further embodiment of this method, the composition can be incorporated into a carrier that comprises one of: a toothpaste, a mouth rinse, a dental floss, a chewing gum, and a lozenge, and the carrier can be administered into the oral cavity to deliver the composition into the oral cavity of the subject. In one such example, in addition to the composition, the carrier can additionally include an active ingredient selected from the group consisting of: sodium fluoride, stannous fluoride, sodium monofluorophosphate, and a combination thereof.

Another embodiment disclosed herein includes a bioengineered bacterium. The bioengineered bacterium embodiment includes a bacterium from the genus Streptococcus containing therein a cell surface protein antigen I/II, spaP gene capable of inducing production of a biofilm, and the bacterium is further bioengineered by introducing spxB gene to be capable of increasing or stabilizing a pH level above 5.5.

In a further embodiment of this bioengineered bacterium, the bacterium is of the species Streptococcus oligofermentans.

In a further embodiment of this bioengineered bacterium, the biofilm adheres to a tooth tissue surface.

In a further embodiment of this bioengineered bacterium, cell surface protein antigen I/II, spaP and spxB genes are introduced via a DNA pFW5 vector.

An additional embodiment disclosed herein includes a bioengineered bacterium. This bioengineered bacterium embodiment includes a bacterium from the genus Lactobacillus containing therein a mucus-adhesion promoting protein capable of inducing adherence of a biofilm containing the bacterium to a surface.

In a further embodiment of this bioengineered bacterium, the bacterium is of the species Lactobacillus reuteri.

In a further embodiment of this bioengineered bacterium, the mucus-adhesion promoting protein gene is introduced via pTRKH3-MapA.

In a further embodiment of this bioengineered bacterium, the biofilm adheres to a gingival tissue surface.

Another embodiment disclosed herein includes a method of bioengineering a bacterium. This method embodiment includes providing a bacterium that is capable of producing a biofilm; and incorporating a surface binding protein gene into the bacterium to cause the bacterium to express a surface modified protein that is adapted to adhere the bacterium to a substrate.

In a further embodiment of this method, the bacterium is selected from the genus Streptococcus, the surface binding protein gene includes a cell surface protein antigen I/II, spaP gene, and, when the bacterium is adhered to the substrate, the bacterium is adapted to increase or stabilize a pH level at the substrate above 5.5 using spxB genes. For example, the bacterium selected from the genus Streptococcus can be a bacterium capable of producing hydrogen peroxide to increase or stabilize the pH level at the substrate above 5.5.

In a further embodiment of this method, the bacterium is selected from the genus Lactobacillus, such as Lactobacillus reuteri, to help prevent periodontitis, for instance, by adhering to tissue of interest and thereby out compete harmful bacteria strains that could otherwise adhere to that tissue of interest.

While multiple embodiments are disclosed, still other embodiments of the disclosure will become apparent to those skilled in the art from the following detailed description, which shows and describes illustrative embodiments of the disclosure. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

The following drawings are illustrative of particular examples of the present disclosure and therefore do not limit the scope of the disclosure. The drawings are intended for use in conjunction with the explanations in the following detailed description wherein like reference characters denote like elements. Examples of the present disclosure will hereinafter be described in conjunction with the appended drawings.

FIG. 1 is a schematic diagram of an exemplary embodiment of an oral biofilm.

FIG. 2. is a flow diagram of an embodiment of a method of altering an oral biofilm to improve dental health.

FIG. 3. is a schematic diagram showing a FW5 vector_spaP gene, a FW5 vector_spxB gene, and a STM, STO and multi-species pH cycling.

FIG. 4 is a representative PCR gel of genes expression spaP and spxB genes into S. oligofermentans (Lanes 2-4; lanes 8-9) & Map gene into L. reuteri (Lane 16-17).

FIG. 5 illustrates resultant biofilm formation for wild and bioengineered S. oligoferementans using a 96 well plate.

FIG. 6 is another illustration of the resultant biofilm formation, as in FIG. 5.

FIG. 7 is a schematic diagram illustrating a method of a biofilm formation assay for Streptococcus oligofermentans.

FIG. 8 illustrates a growth inhibition of S. mutans by bioengineered S. oligofermentans.

FIG. 9 illustrates growth inhibition of S. mutans (right) by bioengineered S. oligofermentans (left) through different pH cycling.

FIG. 10 is a schematic chart showing growth inhibition of S. oligofermentans (STO) when inoculated first through different pH cycling with S. mutans (STM).

FIG. 11 is a schematic chart showing viable cell counts and hydrogen peroxide (HP) production of S. oligofermentans alone and together with S. mutans after 4 hours and 8 hours of incubation.

FIG. 12 illustrate hydrogen peroxide (HP) produced by bioengineered STO, wild STO and S. mutans.

FIG. 13 is a schematic diagram illustrating a method of a biofilm formation assay for dual and multi-species bacteria.

FIG. 14. is a schematic diagram showing a rate of growth of Streptococcus mutans at varying pH levels and sequence of inoculation.

FIG. 15 illustrates an iTero Element II scan of a tooth demonstrating a bioengineered S. oligofermentans treatment effectiveness in killing biofilm forming S. mutans and modifying an acidogenicity of a biofilm.

FIG. 16 is a schematic diagram illustrating a pTRKH3 Vector-MapA, Bioengineered L. reuteri attached to human primary gingival fibroblast (HGF), and a Biofilm production of L. reuteri using four substances including: sucrose, glucose, maltose, and xylitol.

FIG. 17 illustrates the adherence of L. reuteri to dextranomer microspheres via scanning electron microscopy (SEM) imaging.

### DETAILED DESCRIPTION

The foregoing and other features and advantages of the disclosure are apparent from the following detailed description of exemplary embodiments, read in conjunction with the accompanying drawings. The detailed description and drawings are merely illustrative of the disclosure rather than limiting, the scope of the disclosure being defined by the appended claims and equivalents thereof.

Embodiments of the disclosure will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the disclosure. Furthermore, embodiments of the disclosure may include several novel features, no single one of which is solely responsible for its desirable attributes, or which is essential to practicing the disclosure described herein.

The use of the terms “a” and “an” and “the” and similar referents in the context of describing the disclosure are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of about one log from the stated numerical value. For example, the term “about” may be construed to indicate a deviation of between 0.1 to 10 times the stated value. The use of any and all examples, or exemplary language (“e.g.” or “such as”) provided herein, is intended merely to better illuminate the disclosure and does not pose a limitation on the scope of the disclosure unless otherwise claimed. No language in the specification should be construed as indicating any nonclaimed element as essential to the practice of the disclosure.

References to “one embodiment,” “an embodiment,” “example embodiment,” “various embodiments,” etc., may indicate that the embodiment(s) of the disclosure so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The expression “probiotics” is referred to herein as a composition which comprises probiotic microorganisms. Probiotic bacteria are defined as live bacteria, which when administered in adequate amounts confer a health benefit on the host. Probiotic microorganisms have been defined as “Live microorganisms which when administered in adequate amounts confer a health benefit on the host” (FAO/WHO 2002).

The expression “prebiotic” is referred to a composition or a component of a composition which is selectively utilized by host microorganisms conferring a health benefit”. Prebiotics are generally non-viable food components that are specifically fermented in the colon by bacteria thought to be of positive value, e.g., bifidobacteria, lactobacilli, and other short-chain fatty acid producing microorganisms. Prebiotics are also known as an ingredient that allows specific changes, both in the composition and/or activity in the gastrointestinal microbiota that confers benefits upon host well-being and health. The combined administration of a probiotic strain with one or more prebiotic compounds, when designed optimally, may enhance the growth of the administered probiotic in vivo resulting in additional or more pronounced health benefits, and is termed a synbiotic. A synbiotic is formally defined as “a mixture, comprising live microorganisms and substrate(s) selectively utilized by host microorganisms, that confers a health benefit on the host”. Well characterized prebiotics include, for example, galactooligosaccharide (GOS), fructooligosaccharide (FOS), and inulin. GOS and FOS refer to a group of oligomeric, non-digestible carbohydrates that are often produced from monomers using glycosidases to catalyze transgalactosylation reactions. These carbohydrates are often recalcitrant to digestion by host-secreted enzymes in the small intestine, such that they reach the colon intact and are available to the colonic microbiota. It would be understood by those skilled in the art that other compounds that fall within the definition of a prebiotic also can be used in the methods described herein.

As used herein, a “subject” can refer to a human or a non-human. Representative non-human subjects include, without limitation, livestock (e.g., swine, cow, horse, goat, and sheep), poultry (e.g., fowls such as chicken and turkey), and companion animals (e.g., pets such as dogs and cats).

In a further embodiment, the composition further comprises a pharmaceutically acceptable carrier. As used herein, the term “pharmaceutically acceptable carrier” means one or more solid or liquid filler diluents or encapsulating substances which are suitable for administration to a human or an animal and which is/are compatible with the bacterium. The term “compatible” relates to components of the pharmaceutical composition which are capable of being comingled with the further described herein, or a mutant strain thereof in a manner enabling no interaction that would substantially reduce the probiotic efficacy of the organisms selected for the present disclosure under ordinary use conditions. Pharmaceutically acceptable carriers must be of a sufficiently high purity and a sufficiently low toxicity to render them suitable for administration to humans and animals being treated.

A bacterial “strain” as used herein refers to a bacterium which remains genetically unchanged when grown or multiplied. The multiplicity of identical bacteria is included. “Wild type strain” refers to the non-mutated form of a bacterium, as found in nature. In the present context, the term “bioengineered bacterium” should be understood as a strain or bacterium derived from a mother strain by means of e.g., genetic engineering, normal laboratory and commercial production culturing, radiation and/or chemical treatment, and/or selection, adaptation, screening, etc. In specific embodiments the bioengineered bacterium is a functionally equivalent mutant, e.g., a mutant that has substantially the same, or improved, properties (e.g., regarding probiotic properties) as the mother strain. Such a bioengineered bacterium is a part of the present disclosure.

### Compositions

The present disclosure provides for compositions and methods of utilizing engineered probiotic comprising a bacterium for reducing, preventing, or treating oral diseases in a subject. In some aspects, oral diseases may include dental caries, periodontal (gum) disease, or the like. In further aspects, the compositions and methods of the disclosure may be used to reduce, prevent, or treat symptoms and diseases associated with a relatively high acidic environment caused by pathogenic (e.g., dental cavity) causing microbes.

In some embodiments, the composition includes a probiotic. The probiotic may comprise Streptococcus oligofermentans (S. oligofermentans). S. oligofermentans has been considered a potential oral probiotic candidate for caries treatment and prevention. It is frequently isolated from caries-free tooth surfaces and can inhibit the growth of the pathogenic bacterial species Streptococcus mutans in a biofilm, via hydrogen peroxide (HP) production. The mechanism of HP production in S. oligofermentans has been well studied. The levels of HP produced by S. oligofermentans were reported to be sufficient to inhibit S. mutans, but not to hinder its own growth. S. oligofermentans uses 3 types of enzymes to produce HP. The enzymes may include pyruvate oxidase, lactate oxidase (LOX), and L-amino acid oxidase. The ability of S. oligofermentans to convert lactic acid to HP through the LOX enzyme is useful as lactic acid in organic dental biofilms is highly associated with sucrose and/or glucose fermentation, which is known to lead to the formation of dental cavities. Moreover, S. oligofermentans has recently been reported to be capable of inhibiting S. mutans under simulated pathogenic conditions using a high throughput pH-cycling biofilm model. This evidence suggests that S. oligofermentans might be a suitable candidate to compete against pathogenic bacteria including S. mutans in vivo.

Streptococcus oligofermentans bacteria can act to kill pathogenic bacteria present at the oral substrate, such as, for example, Streptococcus mutans. S. oligofermentans bacteria can utilize lactic acid, produced by pathogenic bacteria, such as Streptococcus mutans, to produce hydrogen peroxide which, in turn, can inhibit growth of the lactic acid producing pathogenic bacteria. As a result, binding Streptococcus oligofermentans bacteria at an oral substrate space, such as at the enamel, exposed dentin, cementum, and/or gingival tissue, can act to kill and/or inhibit growth of pathogenic bacteria at the oral substrate space and, thereby, facilitate improved oral health. In other words, adhering Streptococcus oligofermentans bacteria at the enamel, exposed dentin, cementum, and/or gingival tissue via a biofilm can act to place an anti-pathogenic bacteria microbe at the enamel, exposed dentin, cementum, and/or gingival tissue, to thereby help eliminate, and inhibit growth of, pathogenic bacteria at this oral substrate space.

For certain embodiments, the present composition contains a Streptococcus oligofermentans strain engineered to express a cell surface protein gene antigen I/II, spaP gene and putative oxidase known as spxB gene that can cause production of hydrogen peroxide (H₂O₂). The spaP gene is known to have a positive relationship with in vitro biofilm formation. The engineered Streptococcus oligofermentans expresses adhesion factors and has shown the ability to adhere to oral substrates. The adhesion to oral substrates has further been found to have an impact on growth of pathogenic bacteria by occupying the space, thereby “crowding” the pathogenic bacteria out. The spaP and spxB gene sequences are further provided within a sequence listing in XML formats.

In some embodiments, the probiotic may comprise Lactobacillus reuteri (L. reuteri). L. reuteri is a widely distributed intestinal and oral bacterium that is known for its oral health promoting effects. L. reuteri produces antimicrobial molecules, such as organic acids, ethanol, and reuterin. Due to its antimicrobial activity, L. reuteri is able to inhibit the colonization of pathogenic microbes and remodel the microbiota composition in a host. Some L. reuteri strains may reduce the production of pro-inflammatory cytokines while promoting regulatory T cell development and function. As stated, .L. reuteri produces reuterin. Reuterin is an antimicrobial component generated by glycerol fermentation, which has exhibited broad-spectrum antibacterial activity, e.g., proliferative suppression and disruption of biofilm formation on both periodontal pathogens, and caries-inducing Streptococcus mutans. L. reuteri strains are known to have several health-related properties, such as acid-bile tolerance, intestinal adhesion, cholesterol-lowering ability, anti-inflammatory, and antimicrobial properties.

Preferably, the present composition contains Lactobacillus reuteri engineered to express the MapA gene. The MapA gene is a mucus adhesion promoting protein that was cloned in a pTRKH3 plasmid vector and then transformed to L. reuteri. The engineered L. reuteri may adhere to gingival oral tissue to compete with selected pathogens (e.g., Prophyromonas gingivilis). Gene expression cassettes containing the codon optimized adhesion surface protein gene, gene promoters, and terminators were constructed for bacteria strains and cloned. The evaluation of the engineered strain (L. reuteri) was carried out for adhesion using a human cell line from human Primary gingival fibroblast (HGF). The MapA gene sequence is further provided within a sequence listing in XML format.

In some embodiments, the probiotic may comprise one or more of the following microbial strains: Lactobacillus reuteri, Streptococcus oligofermentans, which appear to provide further health promoting effects and inhibit the colonization of pathogenic microbes. The health promoting effects vary under different conditions and from strain to strain.

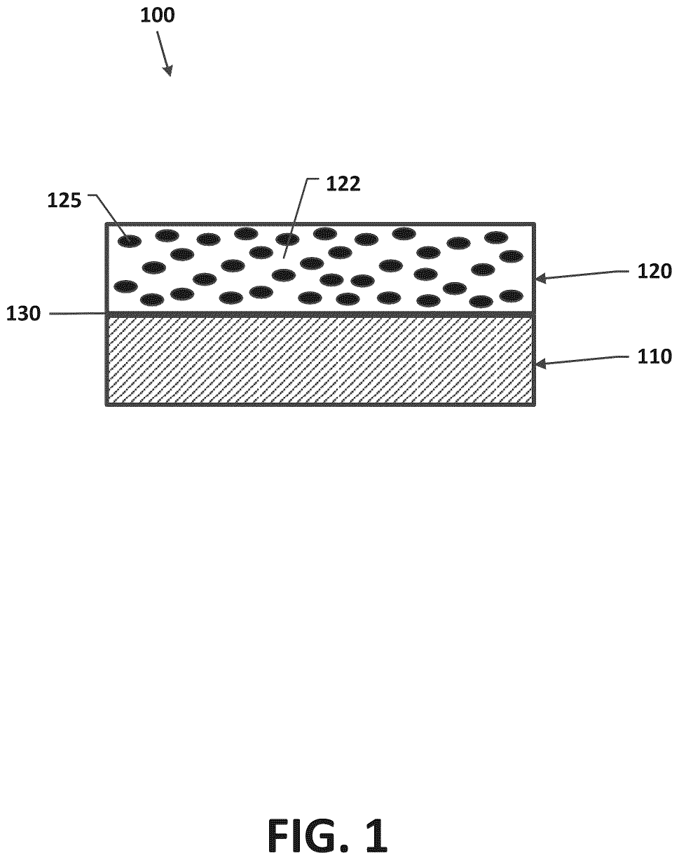

FIG. 1 illustrates a schematic diagram of an exemplary embodiment of an oral biofilm altering device 100. The oral biofilm altering device 100 can be configured to alter a natural oral biofilm state to create an altered oral biofilm state that induces one or more improved oral health properties and/or reduces or prevents instances of malignant bacterium adherence at a same oral cavity substrate where at least a portion of the oral biofilm altering device 100 is adhered.

The oral biofilm altering device 100 can include an oral delivery mechanism 110 and an oral biofilm altering composition 120. The oral biofilm altering composition 120 can be included at the oral delivery mechanism 110. The oral delivery mechanism 110 can be configured to be received at a mouth of a user. For instance, as examples, the oral delivery mechanism 110 can be a toothpaste, a mouthrinse, a dental floss, a chewing gum, or a potable liquid (e.g., water or other beverage). The oral biofilm altering composition 120 can be introduced into the mouth of a user via the oral delivery mechanism 110. The oral biofilm altering composition 120 can be configured to adhere to one or more of enamel, exposed dentin, cementum, and/or gingival tissue, and the oral biofilm altering composition 120 can be configured to alter a composition and at least one property of a biofilm at one or more of the enamel, exposed dentin, cementum, and/or gingival tissue where the oral biofilm altering composition 120 is adhered.

As noted, the oral biofilm altering composition 120 can be included at the oral delivery mechanism 110. In one example, the oral biofilm altering composition 120 can be included at the oral delivery mechanism 110 via a coupling interface 130. When included, the coupling interface 130 can be configured to removably secure the oral biofilm altering composition 120 to the oral delivery mechanism 110. In one such specific example, the oral biofilm altering composition 120 can be included as a coating or layer at the oral delivery mechanism 110 (e.g., as shown in the example of FIG. 1), and the coupling interface 130 can couple the oral biofilm altering composition coating or layer to the oral delivery mechanism substrate. For instance, this example can apply when the oral delivery mechanism 110 is, for example, one of a toothpaste, a dental floss, or a chewing gum. In another such specific example, the oral biofilm altering composition 120 can be dispersed within the oral delivery mechanism 110, and the coupling interface 130 can couple the oral biofilm altering composition 120 to the oral delivery mechanism 110 throughout at least a portion of the to the oral delivery mechanism 110. For instance, this example can apply when the oral delivery mechanism 110 is, for example, one of a toothpaste, a dental floss, or a chewing gum. In an additional such specific example, there need not be a dedicated coupling interface 130 and, instead, the oral biofilm altering composition 120 can be included at the oral delivery mechanism 110 as a mixture of the oral biofilm altering composition 120 and the oral delivery mechanism 110. For instance, this example can apply when the oral delivery mechanism 110 is, for example, one of a mouthrinse or a potable liquid where the oral biofilm altering composition 120 is in a powder or liquid form and mixed together with the mouthrinse or potable liquid oral delivery mechanism 110.

The oral biofilm altering composition 120 can include at least one oral hygiene microbe 122 and at least one binding element 125. The at least one binding element 125 can be configured to adhere the at least one oral hygiene microbe 122 to one or more of enamel, exposed dentin, cementum, and/or gingival tissue. While the schematic diagram of FIG. 1 illustrates the binding elements 125 as dispersed throughout the oral biofilm altering composition 120, embodiments within the scope of this disclosure can have the binding elements 125 disposed around a perimeter of an external surface of the oral biofilm altering composition 120. For some examples, the at least one oral hygiene microbe 122 can be configured to alter a composition and at least one property of a biofilm at one or more of the enamel, exposed dentin, cementum, and/or gingival tissue where the at least one oral hygiene microbe 122 is adhered via the at least one binding element 125. Accordingly, when the oral delivery mechanism 110 is received at a mouth of a user, the at least one binding element 125 can be configured to adhere the at least one oral hygiene microbe 122 to one or more of enamel, exposed dentin, cementum, and/or gingival tissue, and the at least one oral hygiene microbe 122 can be configured to alter a composition and at least one property of a biofilm at one or more of the enamel, exposed dentin, cementum, and/or gingival tissue where the at least one oral hygiene microbe 122 is adhered via the at least one binding element 125. In other examples, the at least one oral hygiene microbe 122 can be one or more benign, non-pathogenic bacterium that are configured to adhere, via the at least one binding element 125, to an oral cavity substrate, such as one or more of the enamel, exposed dentin, cementum, and/or gingival tissue. Accordingly, when the oral delivery mechanism 110 is received at a mouth of a user, the at least one binding element 125 can be configured to adhere the at least one oral hygiene microbe 122 to one or more of enamel, exposed dentin, cementum, and/or gingival tissue, and the at least one oral hygiene microbe 122 can be configured to occupy oral cavity substate space, with one or more benign bacterium bioengineered to adhere to the noted oral cavity substrate(s), that could otherwise be occupied pathogenic bacterium. This can thus help to “crowd out” and thereby prevent instances of pathogenic bacterium adherence at the oral cavity substrate to in turn help improve oral cavity health.

The oral hygiene microbe 122, included in the oral biofilm altering composition 120, can include one or more various types of microbes. The oral hygiene microbe 122 can include one or more types (e.g., different types) of oral hygiene microbes that facilitate favorable biofilm species interactions tailored to the host, environment, and/or metabolic capacity of the oral biofilm at a user's mouth so as to alter the oral biofilm to have improved oral health (e.g., oral hygiene) properties.

As one example, the oral hygiene microbe 122, included in the oral biofilm altering composition 120, can include Streptococcus oligofermentans microbes and Lactobacillus reuteri microbes. In some embodiments, the oral hygiene microbe 122 can include Streptococcus oligofermentans, in other embodiments the oral hygiene microbe 122 can include Lactobacillus reuteri, and in yet other embodiments the oral hygiene microbe 122 can include Streptococcus oligofermentans and Lactobacillus reuteri. Streptococcus oligofermentans, Lactobacillus reuteri, are thought to include properties that reduce incidence of dental caries, and thus, introducing one or both of these microbes as the oral hygiene microbe 122, included in the oral biofilm altering composition 120, may improve oral hygiene by reducing instances of dental caries. In particular, adhering one or both of Streptococcus oligofermentans microbes and/or Lactobacillus reuteri microbes at the enamel, exposed dentin, cementum, and/or gingival tissue via the at least one binding element 125 can help to induce prolonged periods of improved oral health as compared to conventional oral hygiene practices.

As stated, the oral hygiene microbe 122, included in the oral biofilm altering composition 120, can include Lactobacillus genus microbes. Lactobacillus genus microbes can be generally inert bacterium that outcompete pathogenic bacteria at oral substrate space, such as at the enamel, exposed dentin, cementum, and/or gingival tissue. As a result, binding these one or more inert Lactobacillus genus oral hygiene microbes at oral substrate space, such as at the enamel, exposed dentin, cementum, and/or gingival tissue, can prevent or reduce instances of pathogenic bacteria binding at the oral substrate space and, thereby, facilitate improved oral health. In other words, adhering Lactobacillus genus oral hygiene microbes at the enamel, exposed dentin, cementum, and/or gingival tissue via the at least one binding element 125 can act to place an inert oral hygiene microbe at the oral substrate space so as to take up oral substrate space, such as at the enamel, exposed dentin, cementum, and/or gingival tissue, to thereby help to prevent pathogenic bacteria from adhering at this same oral substrate space. Further, Lactobacillus genus microbes, such as L. reuteri have shown to reduce markers of inflammation and thereby aid in preventing periodontal disease,

As stated, the oral hygiene microbe 122, included in the oral biofilm altering composition 120, can include Streptococcus oligofermentans microbes. Streptococcus oligofermentans microbes can act to kill pathogenic bacteria present at the oral substrate. In particular, Streptococcus oligofermentans microbes can utilize lactic acid, produced by pathogenic bacteria, such as Streptococcus mutans, to produce hydrogen peroxide which, in turn, can inhibit growth of the lactic acid producing pathogenic bacteria. As a result, binding Streptococcus oligofermentans microbes at oral substrate space, such as at the enamel, exposed dentin, cementum, and/or gingival tissue, can act to kill and/or inhibit growth of pathogenic bacteria at the oral substrate space and, thereby, facilitate improved oral health. In other words, adhering Streptococcus oligofermentans microbes at the enamel, exposed dentin, cementum, and/or gingival tissue via the at least one binding element 125 can act to place an anti-pathogenic bacteria microbe at the enamel, exposed dentin, cementum, and/or gingival tissue, to thereby help eliminate, and inhibit growth of, pathogenic bacteria at this oral substrate space.

As a further example, the oral hygiene microbe 122, included in the oral biofilm altering composition 120, can include two or more of Streptococcus oligofermentans microbes, Lactobacillus reuteri microbes, Streptococcus gordonii microbes, Actinomyces naeslundii microbes, and/or Prophyromonas gingivilis microbes. For instance, the oral hygiene microbe 122 can be a combination (e.g., mixture) of Streptococcus oligofermentans microbes and Streptococcus gordonii microbes. In another instance, the oral hygiene microbe 122 can be a combination (e.g., mixture) of Streptococcus oligofermentans microbes and/or Streptococcus gordonii microbes plus Lactobacillus reuteri microbes and Actinomyces naeslundii microbes. In yet another instance, the oral hygiene microbe 122 can be a combination (e.g., mixture) of Streptococcus oligofermentans microbes and Streptococcus gordonii microbes plus Lactobacillus reuteri microbes. In yet another instance, the oral hygiene microbe 122 can be a combination (e.g., mixture) of Streptococcus oligofermentans microbes and Streptococcus gordonii microbes plus Prophyromonas gingivilis microbes. In yet another instance, the oral hygiene microbe 122 can be a combination (e.g., mixture) of Lactobacillus reuteri genus microbes and Streptococcus oligofermentans microbes. As noted previously, in some cases, the particular type of microbe(s) used as the oral hygiene microbe 122, included in the oral biofilm altering composition 120, can be selected to facilitate favorable biofilm species interactions tailored to the host, environment, and/or metabolic capacity of the oral biofilm so as to alter the oral biofilm to have improved oral health properties.

The binding element 125, included in the oral biofilm altering composition 120, can include one or more various types of binding elements. The one or more binding elements used for the binding element 125 can be configured to adhere the at least one oral hygiene microbe 122 to one or more of enamel, exposed dentin, cementum, and/or gingival tissue.

As one example, the binding element 125, included in the oral biofilm altering composition 120, can include one or more types of protein that has been engineered to be expressed via the selected microbe 122. The one or more types of protein may be configured to adhere the at least one oral hygiene microbe 122 to one or more of enamel, exposed dentin, cementum, and/or gingival tissue.

One exemplary type of binding protein expressed via the selected microbe (e.g., S. oligofermentans) may be a cell surface protein antigen I/II, spaP gene. In another example, a mucus-adhesion protein MapA may be expressed via the selected microbe (e.g., L. reuteri). In another example, a binding protein for use as the binding element 125 can be surface proteins of the antigen I/II adhesin family, SspA and SspB. These surface proteins of the antigen I/II adhesin family, SspA and SspB, can bind (e.g., directly) to β1 integrin for adhesion at enamel, exposed dentin, cementum, and/or gingival tissue. Moreover, it has been found that these surface proteins of the antigen I/II adhesin family, SspA and SspB, can be compatible with one or more of the types of oral hygiene microbes 122 described previously. As such, use of these surface proteins of the antigen I/II adhesin family, SspA and SspB, as a type of binding element 125 can be useful in complementing the use of the described types of oral hygiene microbes 122 to preserve the function of the described types of oral hygiene microbes 122 while also facilitating adhesion of the oral hygiene microbe 122 at enamel, exposed dentin, cementum, and/or gingival tissue.

Specific types of surface proteins of the antigen I/II adhesin family of binding protein for use as the binding element 125 can include surface lectin proteins and/or salivary amylase proteins. Surface lectin proteins and salivary amylase proteins can be useful in facilitating diverse adhesion via the property of these proteins to recognize different salivary receptors present at enamel surfaces. As such, in some embodiments, it can be useful to include both surface lectin proteins and salivary amylase proteins as the binding element 125 to facilitate a wider array of adhesion capability across different salivary receptors present at enamel surfaces to thereby increase the uptake of the oral hygiene microbe 122 at desired oral substrates.

Another exemplary such type of binding protein for use as the binding element 125 can be a sialic acid binding protein Hsa. The sialic acid binding protein Hsa can be configured to facilitate surface adhesion at enamel, exposed dentin, cementum, and/or gingival tissue. And, like the prior binding protein example, it has been found that the sialic acid binding protein Hsa can be compatible with one or more of the types of oral hygiene microbes 122 described previously. As such, use of the sialic acid binding protein Hsa as a type of binding element 125 can be useful in complementing the use of the described types of oral hygiene microbes 122 to preserve the function of the described types of oral hygiene microbes 122 while also facilitating adhesion of the oral hygiene microbe 122 at enamel, exposed dentin, cementum, and/or gingival tissue.

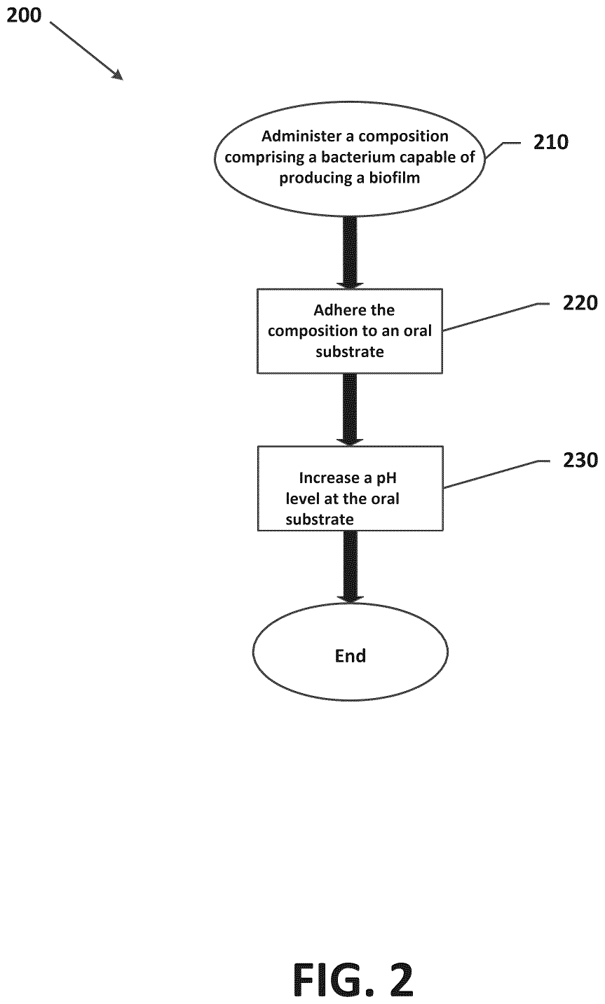

FIG. 2 illustrates a flow diagram of an embodiment of a method 200 of treating an oral cavity of a subject to reduce or prevent instances of dental caries at one or more substrates at the oral cavity. The method 200 can include use of one or more oral delivery mechanisms similar to, or the same as, that disclosed elsewhere herein with respect to the oral delivery mechanism 110, and the method 200 can include use of one or more oral biofilm altering compositions similar to, or the same as, that disclosed elsewhere herein with respect to the oral biofilm altering composition 120.

At step 210, the method 200 includes administering a composition comprising a bacterium capable of producing a biofilm. This bacterium may be selected from: Streptococcus oligofermentans microbes and Lactobacillus reuteri microbes. The altering device can include an oral delivery mechanism, at least one oral hygiene microbe, and at least one binding element. The binding element may include one of a cell surface protein antien I/II, spaP gene expression and/or a MapA gene expression. The at least one oral hygiene microbe and the at least one binding element can be included at the oral delivery mechanism. For example, the oral biofilm altering device placed within the mouth of the user at step 210 can be similar to, or the same as, the oral biofilm altering device 100 described elsewhere herein.

At step 220, when the oral delivery mechanism is placed at the mouth of a user, the method 200 includes the step of adhering the composition to an oral substrate, such as, one or more of enamel, exposed dentin, cementum, and/or gingival tissue using the at least one binding element. For example, the at least one oral hygiene microbe adhered to one or more of enamel, exposed dentin, cementum, and/or gingival tissue at step 220 can be similar to, or the same as, the at least one oral hygiene microbe 122 described elsewhere herein, and the at least one bind element used to facilitate the adhesion of the at least one oral hygiene microbe can be similar to, or the same as, the at least one binding element 125 described elsewhere herein. At step 220, as the at least one binding element (e.g., biofilm) binds to one or more of enamel, exposed dentin, cementum, and/or gingival tissue it can cause the accompanying at least one oral hygiene microbe to adhere to this same enamel, exposed dentin, cementum, and/or gingival tissue.

At step 230, when the at least one oral hygiene microbe is adhered to one or more of enamel, exposed dentin, cementum, and/or gingival tissue, the method 200 includes the step of increasing or stabilizing a pH level at the oral substrates at the oral cavity, above 5.5. In some cases, at step 230, altering a composition and at least one property of a biofilm at one or more of the enamel, exposed dentin, cementum, and/or gingival tissue using the adhered at least one oral hygiene microbe can include utilizing lactic acid, produced by pathogenic bacteria, such as Streptococcus mutans, to produce hydrogen peroxide which, in turn, can inhibit growth of the lactic acid producing pathogenic bacteria and raise a pH level at the oral substrate. In other additional or alternative cases, at step 230, altering a composition and at least one property of a biofilm at one or more of the enamel, exposed dentin, cementum, and/or gingival tissue using the adhered at least one oral hygiene microbe can include use of one or more oral hygiene microbes that are generally inert to outcompete pathogenic bacteria at oral substrate space such that binding these one or more inert oral hygiene microbes at oral substrate space, such as at the enamel, exposed dentin, cementum, and/or gingival tissue, can prevent or reduce instances of the pathogenic bacteria binding at the oral substrate space and, thereby, facilitate improved oral health. In other additional or alternative cases, at step 230, altering a composition and at least one property of a biofilm at one or more of the enamel, exposed dentin, cementum, and/or gingival tissue using the adhered at least one oral hygiene microbe can include use of one or more oral hygiene microbes that kill certain oral pathogenic bacteria but preserve other, health conducive oral microbes so as to selectively and efficiently alter the oral biofilm in a way that improves oral health properties of the oral biofilm.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. However, those of skill in the art should, in light of the present disclosure, appreciate that many changes can be made in the specific embodiments which are disclosed and still obtain a like or similar result without departing from the spirit and scope of the disclosure.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

In one example, S. oligofermentans is considered to be a probiotic candidate for caries as it inhibits the growth of pathogenic S. mutans in biofilms in vitro. Production of hydrogen peroxide (H₂O₂) by S. oligofermentans is one of the mechanisms of action inhibiting S. mutans. Thus, in this example, we genetically engineered S. oligofermentans to improve the adhesion and H₂O₂ production, leading to the inhibition of S. mutans. We chose spaP and spxB genes to enhance adhesion and H₂O₂ production, respectively.

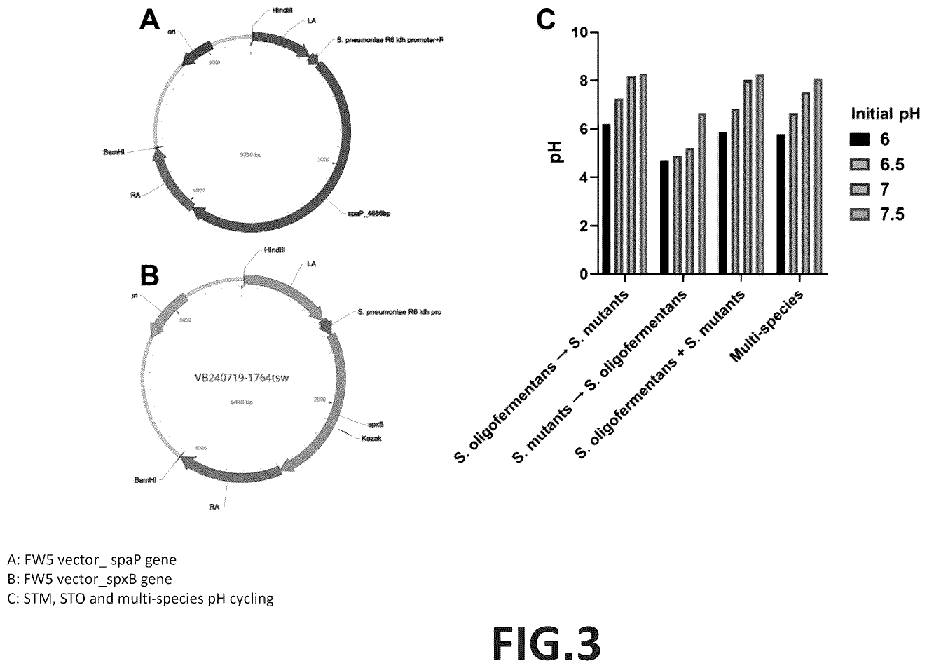

The selected genes were inserted into bacterial cells using a DNA pFW5 vector. Briefly, S. oligofermentans was preconditioned in competence media (10 ml THY supplemented with 500 μl of 4% BSA, 100 μl of 20% glucose, and 20 μl of 10% CaCl2) by culturing cells to mid-log phase (OD600˜ 0.2) at 37° C. with 5% CO2. Preconditioned cultures were diluted 1:100 in fresh competence media and incubated with 2 μl of 0.1 mg/ml competence-stimulating peptide-1 (Csp-1) for 12 min in a 37° C. water bath. Donor DNA pFW5 vector carrying spaP and spxB genes, shown in FIG. 3, extracted from E. Coli was prepared using Quick Plasmid Miniprep Kit (Qiagen). For the gene insertion, a 500-ng genomic was added to 100 μl of stimulated cells and incubated in a 30° C. water bath for 30 min. After allowing contact of exogenous DNA and bacteria, mixtures were diluted 1:100 in fresh competence media and allowed to expand in a 37° C. water bath for 2 hrs. before plating on spectinomycin plates. Plates were incubated overnight at 37° C. with 5% CO₂ to select transformants.

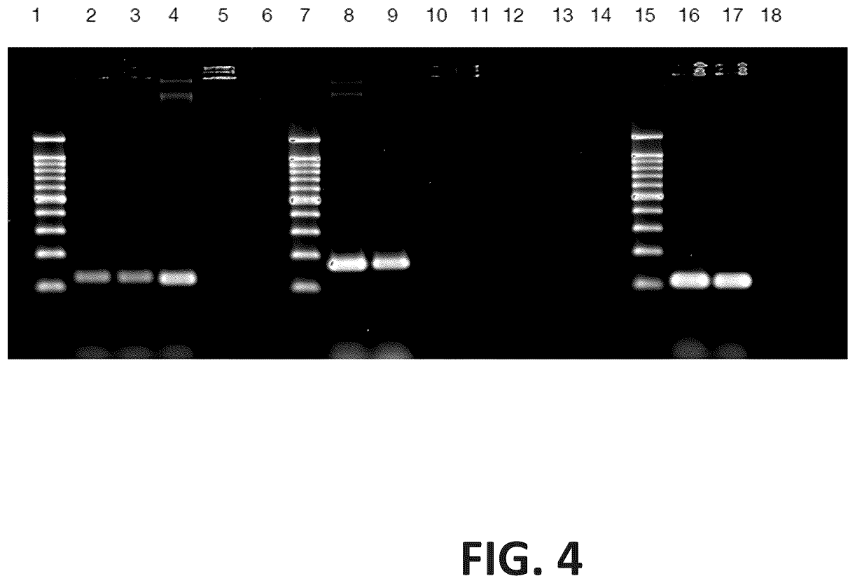

FIG. 4 is a representative PCR gel of gene expression for spaP and spxB genes inserted into S. oligofermentans. Shown in lane 1: DNA marker (100 bp ladder); lane 2: positive control (spaP gene); lane 3: S. mutans; lane 4: bioengineered S. oligofermentans; lane 5: negative control (wild S. oligofermentants); lane 7: DNA marker (100 bp ladder); lane 8: positive control (spxB gene); lane 9: bioengineered S. oligofermentans (spxB gene); lane 10: negative control (S. mutans); lane 15: DNA marker (100 bp ladder); lane 16: positive control; lane 17: bioengineered L. reuteri (MapA gene); lane 18: negative control.

The expression of the spaP gene into S. oligoferementans gave the expected band size of 121 bp (100% similarity; accession numbers X17390). The expression of the spxB gene into S. oligoferementans gave the expected band size using universal primers spxB F/R (100% similarity; accession numbers VTT23904).

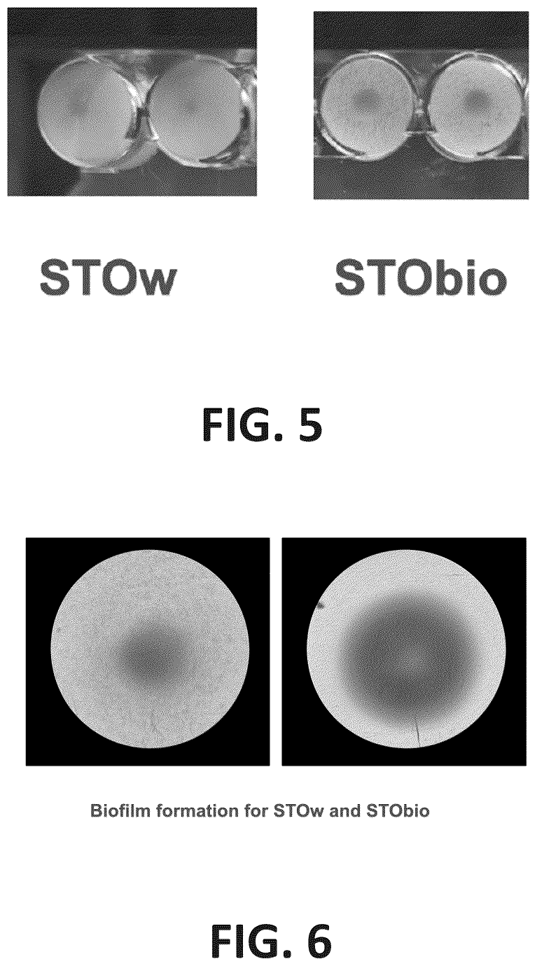

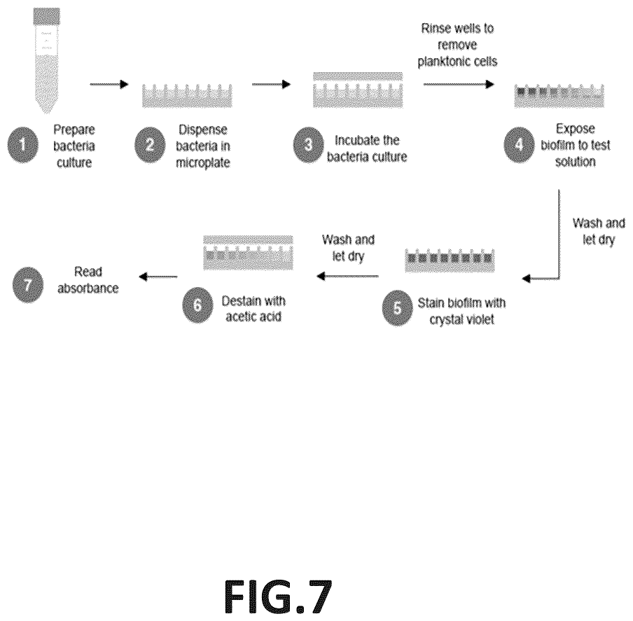

A quantitative comparison between wild and bio-engineered Streptococcus oligofermentans in biofilm formation was carried out by the inoculation of single colonies from 24 h Todd Hewitt medium (THY) plate agar. Single colonies were inoculated in 6 ml of odd Hewitt medium (THY) broth and incubated anaerobically at 37° C. for 18 h. The cultures were collected by centrifugation at 8,000 rpm for 5 min and washed 3 times in 2 ml of PBS buffer. Each pellet was resuspended in THY to yield an optical density value at a wave-length of 600 nm. The cultures were then suspended in fresh media and inoculated in 96 well plates and further incubated for 24 h at 37° C. (FIG. 5, 6). After incubation, media was discarded, and the plates rinsed twice to remove unattached cells and media components. This step was completed to improve accuracy of data collection and significantly lower background staining.

A 125 μL of a 0.1% solution of crystal violet [CV] in water was added to each well of the microtiter plate and then incubated at room temperature for 10-15 min. The plates were rinsed 3-4 times with water by submerging in a tub of water as outlined above, shook out and blotted vigorously to rid the plate of all excess cells and dye. 125 μL of 30% acetic acid in water was added to each well of the microtiter plate to solubilize the CV, and the microtiter plate was incubated at room temperature for 10-15 minutes. A 125 μL of the solubilized CV was transferred to a new flat bottomed microtiter dish and the quantitative absorbance was carried in a plate reader at 600 nm using 30% acetic acid in water as the blank, as shown in FIG. [symbol] (Emery Pharma picture; https://emerypharma.com/solutions/cell-microbiology-services/biofilm-eradication-testing/).

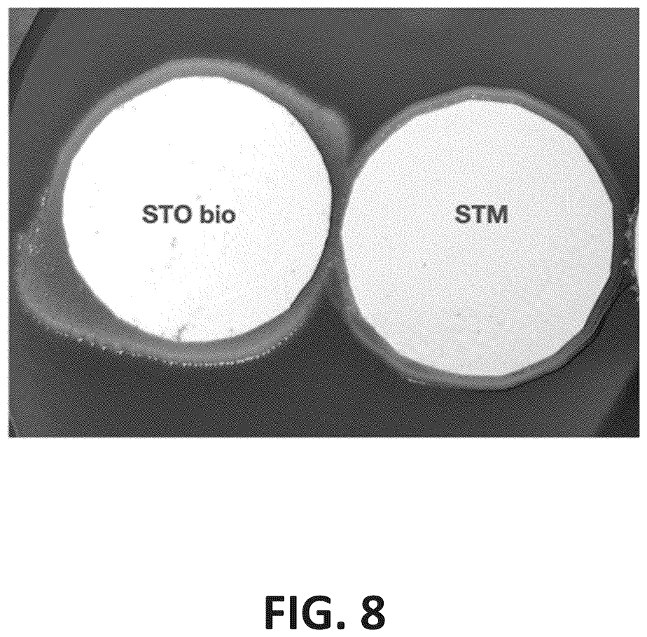

The inhibitory effect of S. oligofermentans is relatively specific to S. mutans, as S. oligofermentans exhibited potent inhibition against S. mutans. We performed an inhibition assay on blood agar plate using discs in a 96-well plate. Each 125 ul (1×108 cells) overnight culture of S. oligofermentans and S. mutans with the same optical density at 600 nm was spotted on sterile disc diffusion, applied on a blood plate and incubated at 37° C. for 24 hrs. The results, shown in FIGS. 8, showed a clear inhibition of S. mutans by bioengineered S. oligofermentans.

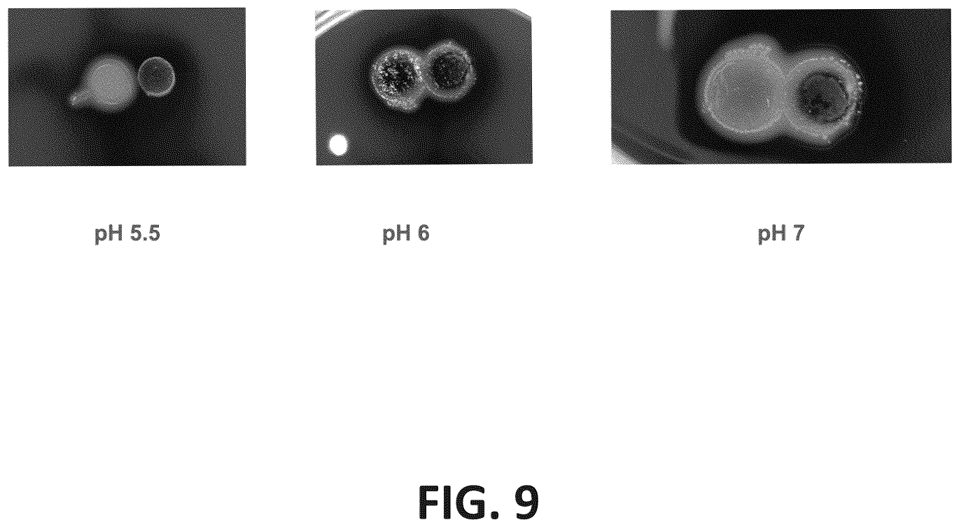

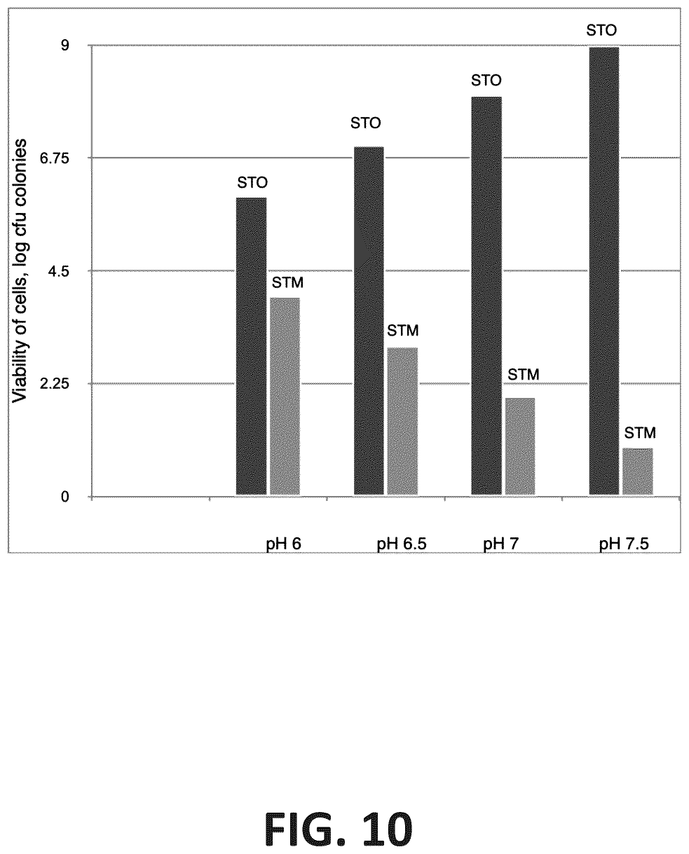

Further, the inhibition of growth of S. mutans by bioengineered S. oligofermentans at different pH values was evaluated. Around 10 ul (1×10⁸ cells) overnight culture of S. oligofermentans and S. mutans with the same optical density at 600 nm was spotted on a blood plate with different pH values and incubated at 37° C. for 24 hrs. The results, as shown in FIGS. 9 and 10, showed at pH 5.5 both S. oligofermentans and S. mutans lost their ability to compete with each other, which may be a result of the poor growth of both strains. At pH 6, S. oligofermentans showed a better performance than pH 5.5. The effect of S. oligofermentans was slightly clear. However, at pH 7, the effect was very clear, which is in accordance with our previous results and related to the H₂O₂-production capacity of S. oligofermentans under different pH conditions.

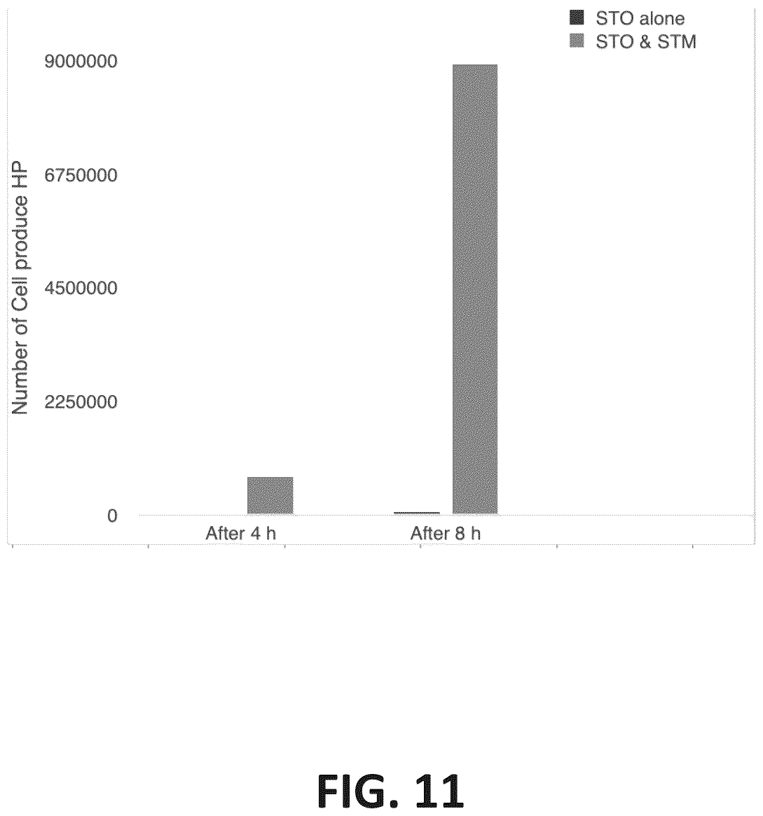

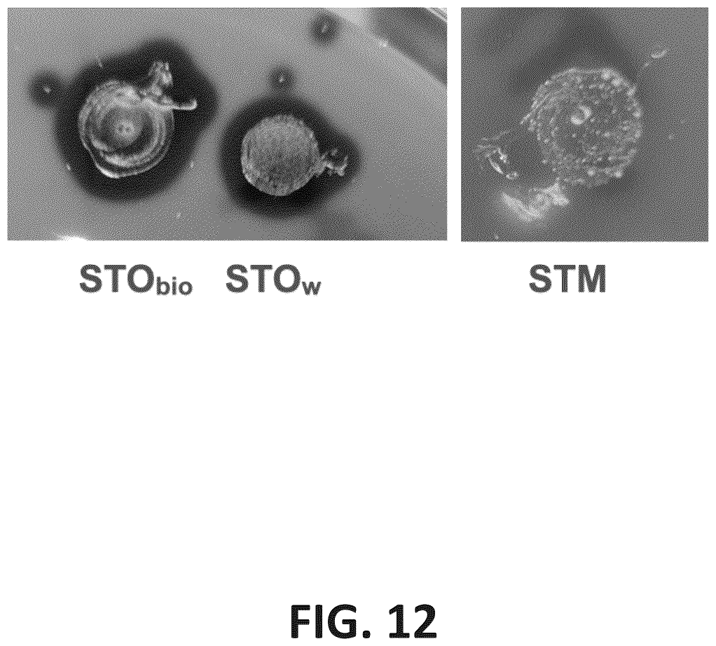

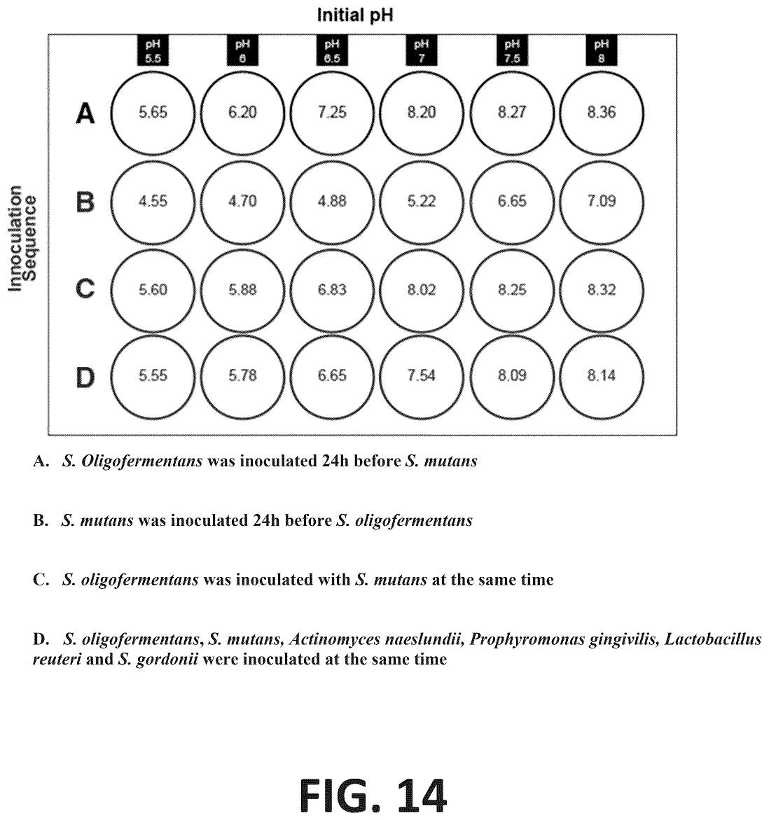

The detection and quantification of the amount of hydrogen peroxide (H₂O₂) generated by S. oligofermentans was evaluated, as shown in FIGS. 11 and 12. Streptococcus oligofermentans generates a sufficient amount of H₂O₂ to inhibit S. mutans. As H₂O₂ was the potential inhibitory substance, we next quantified precisely the amount of H₂O₂ generated by bioengineered and wild S. Oligofermentans. Prussian Blue Agar, was employed to detect H₂O₂ producing oral streptococci. This agar comprised 52 g/L of BHI agar (Acumedia), 1 g/L of iron (III) chloride (Sigma-Aldrich, St. Louis, MO, USA), and 1 g/L of potassium hexacyanoferrate (Sigma). Overnight cultures of oral streptococci (10 ul) with same cell amount (1×10⁸) were spotted on Prussian Blue agar and incubated at 37° C. for 4 hrs. before visualization. The results, shown in FIG. 14, showed that bioengineered S. oligofermentans produced a bigger blue spot compared to the wild strain due to the excess production of H₂O₂, related to inserted spxB genes. On the other hand, S. mutans does not include a blue spot, which confirms that S. mutans did not produce any hydrogen peroxide (H₂O₂).

Artificial saliva (1 ml) was dispensed into each well of a sterile 24-well tissue culture plate. Hydroxyapatite discs (9.6 mm diameter) were sterilized by autoclaving and then transferred aseptically into wells. All wells are also supplemented with 200 μl of fresh saliva. Inoculated plates were mounted onto an orbital shaker and continuously mixed gently for 12 hours at 37° C. Controls comprised wells in which hydroxyapatite discs were incubated with 2 ml of artificial saliva and 200 ml salivary inoculum. The rest of the wells were inoculated with equal numbers of strains in fresh artificial saliva. After 48 hrs. incubation, hydroxyapatite discs were aseptically removed from the wells, gently washed in saline, and then placed in Universal bottles containing 8 ml BHI broth. Additionally, 1 ml of the bulk, planktonic phase was also removed from each well and serially diluted for plate counting. These were immediately vortexed thoroughly for 2 minutes and subjected to differential plate counting as outlined below.

Sterilized enamel blocks were coated with sterile clarified saliva (sHA). Saliva was sterilized by filtration through 0.22 μm syringe filters after centrifugation at 6,000×g for 10 min. Prior to the onset of the biofilm development, colonies from three pathogenic species were picked and inoculated into modified Brain Heart Infusion (MBHI) broth (Brain Heart Infusion broth supplied with 1% sucrose, 1% glucose, 8 g/L meat extract, 10 mM L-arginine, and 250 mM glycerol, pH 7.0, respectively. Pure cultures of each pathogenic strain were collected in the exponential phase, diluted with fresh BHI, and adjusted the turbidity to the optimal optical density at 600 nm (OD600). A volume of 250 μL of the bacterial suspension containing equivalent amounts of each strain was dispensed equally into six independent wells. Initially, pellicle-coated specimens were immersed in the mixed bacterial suspension described above under static condition for 24 hrs. to facilitate bacterial adhesion and biofilm formation. Following the preliminary incubation, equal amounts from each of the probiotic strains was added to each well. Measurement of pH values were monitored every 6 hrs. After 48 hrs., the enamel specimens were carefully taken out with sterile tweezers and washed twice using 1 mL of PBS in 6-well plates to remove loosely bound cells. The adherent biofilms were harvested from specimens by vortexing in sterile tubes and transferred into 1 mL sterile BBS tubes. One hundred microliters of the microbial suspension of each specimen were serially diluted 10-fold after vortex mixing and inoculated on Columbia Agar supplied with 5% sheep blood plates in triplicate. The numbers of colonies on agar plates were counted and determined after the incubation at 37° C. for 48 hrs.

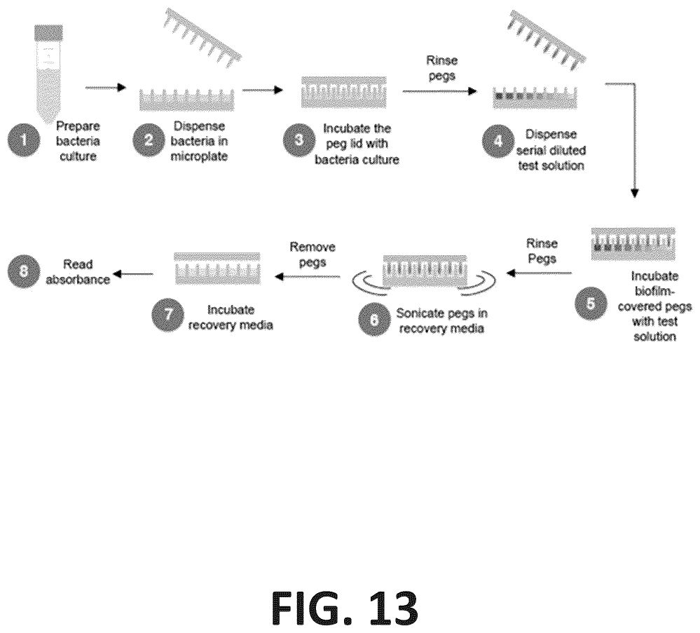

The biofilm cultivation scheme was cultivated in an active attachment model using a 96-well plate supplied with pegs, human teeth samples and hydroxyapatite discs. The general procedure for growing a biofilm was as follows: overnight (16 h) cultures were diluted to a final OD 600 nm of 0.04 in fresh buffer medium with different pH cycling ranges from 5.5 to 8, and 200 μL of this suspension was dispensed into each well of a 96-well plate. The plate was covered with a lid containing pegs to allow the active attachment and growth of biofilms. The 48-hour biofilms formed on pegs were checked for the current pH and collected for viable cell counts (FIG. 13) (Emery Pharma picture; https://emerypharma.com/solutions/cell-microbiology-services/biofilm-eradication-testing/). For multi-species biofilm besides S. oligofermentans and S. mutans the other species that included were Streptococcus gordonii, Actinomyces naeslundii, Prophyromonas gingivilis, and Lactobacillus reuteri.

To investigate the impact of inoculation sequence on the competition between S. mutans and S. oligofermentans, either strain was inoculated as a single-species biofilm and grown for 24 hrs. The second bacterial species was added to the 24-h biofilms and grown for another 24 hrs. All experiments were repeated 3 times, and each experiment included 4 replicates.

The viability of the biofilms was examined by plate counting. In brief, each individual peg with biofilms was cut off with a sterile scalpel and dispersed by sonication on ice 60 times for 1 second at an amplitude of 700 W (Thermo Fisher Scientific™, Model 705 Sonic Dismembrator., USA). Undiluted and serially diluted samples (100 μL) were plated on Brain Heart Infusion agar plates and incubated anaerobically for 3 days. The colony-forming units (CFUs) from each biofilm were counted. The morphologies of S. mutans and S. oligofermentans were distinct on the Brain Heart Infusion agar plates; therefore, the CFUs of S. mutans and S. oligofermentans in the dual-species biofilm samples were counted separately based on their colony morphology. The colonies of S. mutans were whitish and had a textured appearance, whereas the colonies of S. oligofermentans were yellowish and smooth. The detection limit of this viable cell count method is 100 CFUs per species per biofilm.

### Results

After a 48-hour growth period of the same initial cell count of both wild and bio-engineered (mutant) S. oligofermentans, we successfully demonstrated the improved ability of bio-engineered S. oligofermentans to adhere to simulated surfaces (pegs, teeth samples, hydroxyapatite discs) compared to the wild type. Based on our results of biofilm formation on pegs, a total of 4.7×10⁶ of the bio-engineered S. oligofermentans adhered to the surface compared to 3.9×10⁶ for the wild type. Similar results were collected as for pegs for human teeth samples and hydroxyapatite discs. Hence, we successfully improved the adherence rate of the bioengineered S. oligofermentas.

A 24-hour growth period of the mutant of S. oligofermentans and S. mutans under different pH conditions was also investigated. Our results showed that the sequence of inoculation and pH variation resulted in different inhibitory effects. At pH values of 5.5 and 8.0, S. oligofermentans and S. mutans exhibited poor growth and no interaction was observed regardless of the inoculation sequence. At pH values of 6.0, 6.5, 7.0, and 7.5, S. oligofermentans and S. mutans showed interactions that were significantly influenced by the inoculation sequence. When S. mutans was inoculated first, the growth of S. oligofermentans was completely inhibited.

When the two species were inoculated at the same time, or when S. oligofermentans was inoculated first, an inhibitory effect by S. oligofermentans on S. mutans was observed (FIG. 14). The optimal result was observed at pH 7 where we can see very clear the inhibition effect of the mutant S. oligofermentans on the growth of S. mutans. By comparing the results obtained from wild and mutant S. oligofermentans, we could see clearly there is an improvement in the pH value. Our study indicated that the optimal pH for S. oligofermentans growth is pH 7.0.

The adherence rate for the bio-engineered (mutant) S. oligofermentans and S. mutans was also investigated. Our bio-engineered S. oligofermentans showed a strong adherence to the pegs teeth (4.27×10⁶) and hydroxyapatite discs (2×10⁶). However, the adherence of S. mutans (5.2×10⁶; 2.9×10⁶) stayed slightly higher than our bio-engineered S. oligofermentans but still comparable.

The multi-species biofilm included S. oligofermentans and S. mutans, Streptococcus gordonii, Actinomyces naeslundii, Prophyromonas gingivilis, and Lactobacillus reuteri. The results showed that there was a clear improvement in pH value and the optimal result was observed at pH 7 and 7.5, as shown in FIG. 14.

### Example 3

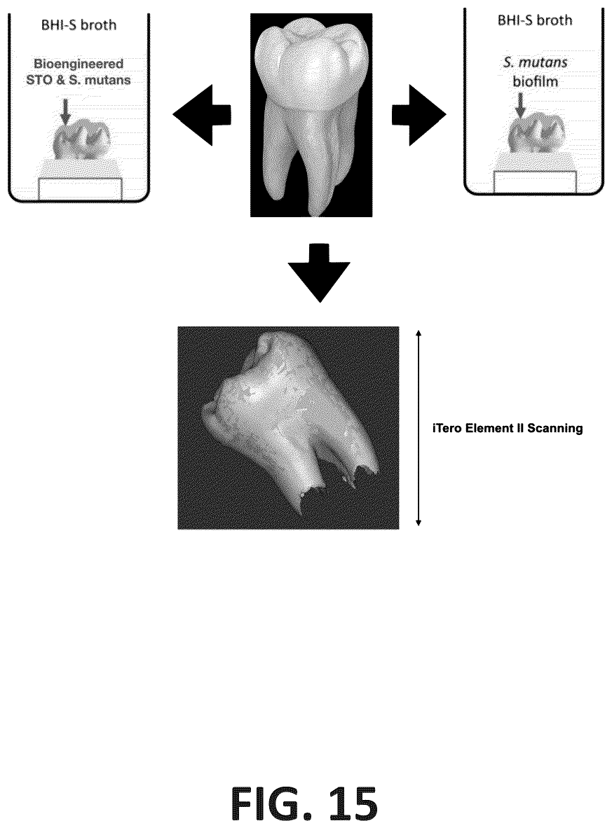

FIG. 15 illustrates the characterization of human molars demineralized by S. mutans biofilm and the effect of the bioengineered S. oligofermentans on tooth demineralization caused by the S. mutans biofilm. In this additional study, the aim was to confirm the effect of S. mutans on the demineralization of human teeth and the role of our bioengineered S. oligofermentans on inhibition of viable S. mutans in the biofilm formed on the tooth surface and thus would prevent or deter the demineralization process caused by bacterial activity.

### Methods and Materials

Human molars without cracks, fractures, caries, or any defects were prepared. These 2 molars were collected, cleaned and autoclaved for sterilization. First, volumetric was recorded using an Itero Element II scanner before the experiment performed. To induce initial caries-like demineralization (hereafter referred to as initial caries), S. mutans biofilms were prepared by culturing S. mutans in modified Brain Heart Infusion (Brain Heart Infusion broth supplied with 1% sucrose, 1% glucose, 8 g/L meat extract, 10 mM L-arginine, and 250 mM glycerol, pH 7.0). Incubation for all experimental steps, including the continuous-culture biofilm model, was conducted at 37° C. under aerobic conditions. Around 1×10⁸ S. mutans cells were loaded in BHI media with the first molar tooth. Similarly, a mix culture of equal volume and cell count (1×10⁸) was performed using S. mutans and bioengineered S. oligofermentans and was incubated at 37° C. under aerobic conditions for 5 days. At the end of the experiment, the scanning of the two teeth was performed in order to carry the comparison between the initial and post volumetric changes.

### Results

The volumetric analysis revealed that this period of incubation with S. mutans resulted in the generation of a radiolucent layer on the outer surface of the tooth. The initial volumetric for the first molar before the experiment was 993.728 cm³. However, after the incubation with S. mutans biofilm, there was a decrease in the volumetric size with 0.07% (993.044 cm³) suggesting demineralization. The presence of S. mutans illustrates that the demineralization of the tooth surface will continue to grow, allowing the acid to work deeper into the tooth to cement the damage. The lactic acid will continue to keep the pH low, further promoting demineralization and resulting in a subsurface lesion. However, in the example where both strains were loaded together, the initial volumetric was 1053.52 cm³, while at the end of the experiment the volumetric size was 1054.17 cm³. Our bioengineered S. oligofermentans includes a strong adhesive and low carbohydrate fermentative capacity and produces hydrogen peroxide by utilizing lactic acid to inhibit pathogens such as S. mutans. The results demonstrated that bioengineered S. oligofermentans treatment was effective not only in killing biofilm forming S. mutans but also in modifying the acidogenicity of the biofilm, and exhibits decolonization capability via displacement modes and thus, it could deter the progression of tooth demineralization.

### Example 4

In another example, L. reuteri electrocompetent cells were prepared by growing 5 ml of culture in MRS at 37° C. with 5% CO₂ until OD 600 nm of ˜1.0. Cells were then pelleted and resuspended in 10 ml of sterile cold 0.5 M sucrose and 10% glycerol twice, followed by a final resuspension in 100 μl sterile cold 0.5 M sucrose and 10% glycerol. To this resuspension, 1 μg of pTRKH3-MapA (MapA: A Mucus Adhesion Promoting Protein) was added and the cell/DNA mixture was placed into an ice cold 2 mm electroporation cuvette (BioRad, Hercules, CA). Cells were electroporated at 2500V, 25 μF and 400Ω using a BioRad Gene Pulser Xcell (BioRad, Hercules, CA). Immediately after electroporation, cells were resuspended in 1 mL of MRS and incubated at 30° C. for 2 hrs., followed by serial dilution and plating onto MRS agar containing 100 μg/ml erythromycin and incubated at 30° C. The positive colonies were confirmed by PCR for targeting the Map A gene. Further, the expression of the MapA gene into L. reuteri gave the expected band size 111 bp using universal primers MapA F/R (100% similarity; accession numbers AJ293860).

To investigate the impact of different nutritious prebiotic substances that contribute to probiotic growth and promote further biofilm production, four substances were compared including: sucrose, glucose, maltose, and xylitol. Water was added as control for comparison. Briefly, overnight bioengineered, and wild L. reuteri strains were grown on MRS agar, and a single colony was taken and incubated in 10 ml of MRS broth containing either: water (control), 1M maltose, 1M sucrose, 1M xylitol, or 1M glucose. To examine bacterial adherence to the human cell line, each culture was treated with fluorescein isothiocyanate (FITC) as a label and examined using a multi-function microplate reader (GloMax Microplate Reader, Promega, MA). Lasers were set at 485 nm for excitation and 530 nm for emission.

Bacterial cells were harvested by centrifugation at 10,000×g and 4° C. for 5 min. Then, we resuspended bacterial cells in 1 ml of a.0.1 M sodium bicarbonate buffer. We measured the optical density at 600 nm from a 1:100 dilution of cell suspension in sodium bicarbonate. We diluted cells to 10¹⁰ cells/mL in a total of 1 mL sodium bicarbonate. A 0.2 μl of FITC stock solution was added to the cell suspension and immediately vortexed. The cells were incubated in the dark with end-over-end rotation for 30 min at room temp. Cells were washed four times with HBSS to remove unbound dye and resuspend in 1 mL of HBSS. The optical density was measured at 600 nm from a 1:100 dilution and resuspend the cells to the desired concentration in either HBSS or medium.

Gingival fibroblast (HGF) cell culture. HGF cells were obtained from the American Type Culture Collection (Manassas, VA, USA). These cells were cultured in Dulbecco's Modified Eagle's Medium (DMEM, Lonza Walkersville, MD, USA) with the addition of 10% Fetal Bovine Serum at 37° C. in a humidified atmosphere of 95% air and 5% CO2. The medium was renewed every second day. Cells were subcultured once a week with a split ratio of 1:3 using 0.25% trypsin-EDTA solution. Images of the cells were captured using a Zeiss microscope (Carl Zeiss, Oberkochen, Germany).

The adhesion assay was determined according to the method of Wang et al. (2014), with minor modifications (Wang et al (2014): Wang, T., H. Sun, J. Chen, L. Luo, Y. Gu, X. Wang, Y. Shan, Y. Yi, B. Liu, Y. Zhou, and X. Lu. 2021. Anti-adhesion effects of Lactobacillus strains on caco-2 cells against Escherichia coli and their application in ameliorating the symptoms of dextran sulfate sodium-induced colitis in mice. Probiotics Antimicrob. Proteins 13:1632-1643. https://doi.org/10.1007/s12602-021-09774-8). A human primary gingival fibroblast (HGF), which was isolated from the gingiva, was obtained from ATCC (Manassas, VA, USA). Cells were seeded in 6-well tissue culture plates (Gibco, New York, NY) at 37° C. under 5% CO₂. The medium in the wells was replaced with fresh medium until a monolayer formed with no more visible differentiation. Before adherence assays, the HGF monolayer was washed twice with sterilized Dulbecco's PBS (DPBS). Lactobacillus strains were grown in MRS broth under anaerobic conditions at 37° C. for 24 hrs. and then labeled with fluorescein isothiocyanate (FITC; Sigma-Aldrich). Subsequently, 1 mL of FITC-labeled Lactobacillus suspension (1×10⁶ cfu/mL in DMEM) and 1 mL of antibiotic-free DMEM were added to each well containing HGF cells and incubated at 37° C. in a 5% CO₂ atmosphere. After 2 hrs. of incubation, cells were washed twice with DPBS and examined using a multi-function microplate reader. Lasers were set at 485 nm for excitation and 530 nm for emission.

A similar experimental protocol to study the exclusion assay was used to analyze competitiveness between wild and bioengineered Lactobacillus. For the competition assays, 1 mL of wild Lactobacillus strain suspension (1×10⁶ cfu/mL) and 1 mL of bioengineered suspension (1×10⁶ cfu/mL) were added simultaneously to each well of HGF cells and incubated at 37° C. in a 5% CO₂ atmosphere and incubated for 4 hrs. Competitiveness was calculated as the percentage adhesion (%) of wild in combination with bioengineered Lactobacillus relative to the wild strain adhesion in the absence of bioengineered Lactobacillus (control).

### Results

In this study we successfully engineered L. reuteri in order to enhance the adherence to human primary gingival fibroblast (HGF). The gene of choice was the MapA gene, which is a mucus adhesion promoting protein. MapA mediates the adhesion of Lactobacillus reuteri to Caco-2 human intestinal epithelial cells. In this study, the bioengineered strain that carries this gene showed an improved adherence to gingival tissue compared to the wild strain (see Table below). Furthermore, the adherence rate for the pretreated strains from both types with maltose, sucrose, xylitol and glucose were nearly three times higher in the engineered strain compared to the wild strain. This is because L. reuteri has extracellular glucosyltransferase (GTF) proteins that catalyze the formation of exopolysaccharides of glucan from disaccharide sugars such as sucrose, maltose, etc. By comparing the pretreated engineered strains and wild strains with the 4 substances, a high adherence was observed for the strains that were treated with maltose and xylitol. Xylitol was slightly higher than maltose for the wild strain. When evaluating the engineered strain, the difference was quite remarkable (see Table below). This suggests that xylitol may be a suitable formulation to pretreat our bio-engineered probiotic before delivery to enhance its adherence to gingival tissues. Further, xylitol has been widely documented to have dental health benefits, such as reducing the risk for dental caries, and it is also commonly used as a sweetener.

| No. of strains | | | | | |
|---|---|---|---|---|---|
| attached to HGF | Control | Glucose | Sucrose | Maltose | Xylitol |
| Wild L. reuteri | 1.29 × 10² | 1.42 × 10² | 2.68 × 10² | 3.88 × 10² | 4.55 × 10² |
| Bioengineered | 5.33 × 10² | 6.33 × 10² | 7.11 × 10² | 8.51 × 10² | 4 × 10³ |
| L. reuteri | | | | | |

### Example 5

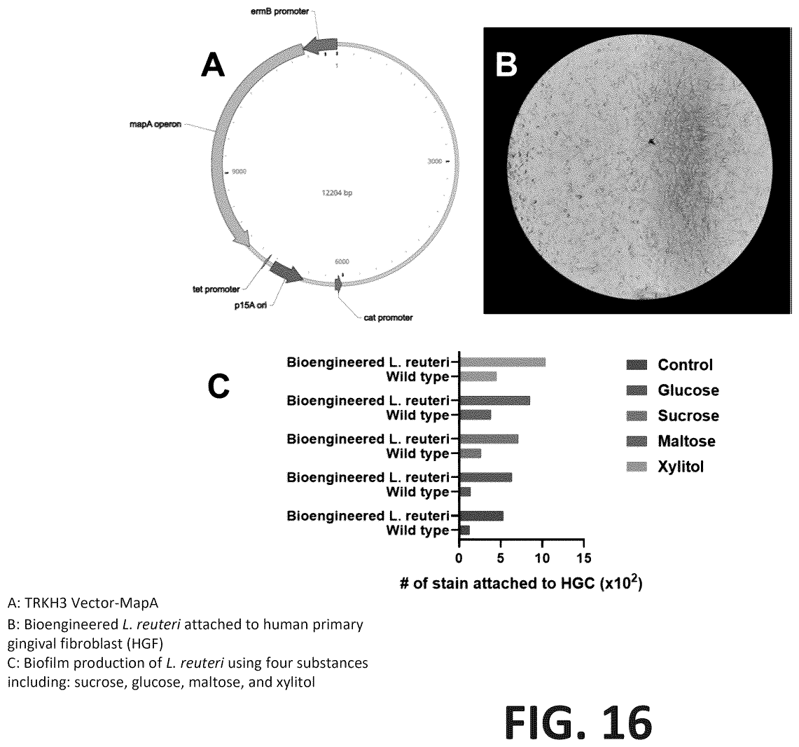

This study looks at bioengineered L. reuteri for targeting periodontitis, and enhancing the biofilm formation and the adherence of the pretreated L. reuteri when delivered on dextranomer microspheres that contain beneficial cargo. FIG. 16 illustrates; A: pTRKH3 Vector-MapA, B: Bioengineered L. reuteri attached to human primary gingival fibroblast (HGF), and C: Biofilm production of L. reuteri using four substances including: sucrose, glucose, maltose, and xylitol.

### Methods and Materials

In this approach, dextranomer microspheres (DMs) (a macroscopic porous microsphere, Sephadex G-25 Superfine, Cytiva, Marlborough, MA) was used as a biocompatible surface to take advantage of L. Reuteri's native ability to bind to this cross-linked dextran at sufficiently high concentrations to enhance L. reuteri's probiotic effects. The DMs were hydrated overnight with a filter sterilized 1 M maltose, 1M sucrose, 1M Xylitol, or 1M glucose solution for overnight at room temperature. The fluorescence bioengineered suspension (1×10⁶ cfu/mL) was prepared as above and incubated with microspheres filled with either: water, 1M maltose, 1M sucrose, 1M fructose, or 1M glucose for 30 mins. To examine bacterial adherence to the microspheres, solutions were combined and incubated for 5 min in a Micro Bio-Spin column (BioRad, Hercules, CA). The columns were then centrifuged (100×g) for 1 min. The flow-through was serially diluted and plated to calculate the total number of non-adhered bacteria, and this value was subtracted from the total number of starting bacteria to derive the total number of adhered bacteria. For all experiments, a control preparation that consisted of bacteria with no microspheres was used. The adhered fluorescence bioengineered L. reuteri to the DMs were added simultaneously to each well of HGF cells and incubated at 37° C. in a 5% CO₂ atmosphere and incubated for 2 hrs.

### Results

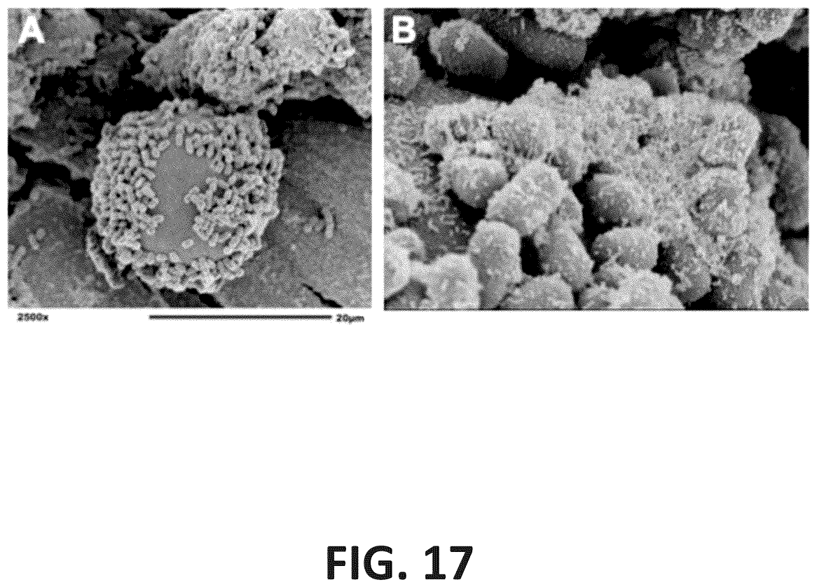

Next, we investigated whether our bioengineered L. reuteri in its biofilm state adhered to DMs would improve adherence to human primary gingival fibroblast (HGF). There was a significant increase in the number of the bioengineered strains to the HGF even when the incubation was just 2 hrs. (see Table below). The DMs that were treated with Xylitol and Maltose showed to have a better biofilm formation for L. reuteri compared to sucrose and water, as shown in FIG. 17 (Al-Hadidi, A., Navarro, J., Goodman, S. D., Bailey, M. T., and Besner, G. E. (2021). Lactobacillus reuteri in its biofilm state improves protection from experimental necrotizing enterocolitis. Nutrients 13, 918). This is because DMs are porous and can be preloaded with nutritious prebiotic substances that contribute to probiotic growth and promote further biofilm production. These results showed that the engineered enhanced L. reuteri adhere to microspheres that are accompanied with beneficial cargo. This shows a very promising and novel therapeutic delivery platform that lays a foundation for its application in other microbial therapeutic delivery candidates and furthers the progress of the L. reuteri delivery system towards human use. FIG. 17 shows the adherence of L. reuteri to dextranomer microspheres (DMs). Scanning electron microscopy (SEM) image demonstrate the adherence of L. reuteri to the surface of a biocompatible DMs (Al-Hadidi et al., 2021).

| No. of strains attached | | | | |
|---|---|---|---|---|
| to HGF using | | | | |
| microspheres (DMs) | Control | Sucrose | Maltose | Xylitol |
| Bioengineered | 4.05 × 10³ | 7.42 × 10³ | 7.92 × 10³ | 8.04 × 10³ |
| L. reuteri | | | | |

While the disclosure has been described in connection with various embodiments, it will be understood that the disclosure is capable of further modifications. This application is intended to cover any variations, uses or adaptations of the disclosure following, in general, the principles of the disclosure, and including such departures from the present disclosure as, within the known and customary practice within the art to which the disclosure pertains.

What is claimed is:

1. A method of treating an oral cavity of a subject to reduce or prevent instances of dental caries at one or more substrates at the oral cavity, the method comprising:
administering into the oral cavity of the subject a composition comprising a bacterium capable of producing a biofilm; and
after administering into the oral cavity the composition comprising the bacterium capable of producing the biofilm, increasing a pH level, at the one or more substrates at the oral cavity, above 5.5.

2. The method of claim 1, wherein the bacterium is selected from Streptococcus oligofermentans and Lactobacillus reuteri.

3. The method of claim 2, wherein the bacterium is Streptococcus oligofermentans.

4. The method of claim 3, wherein the bacterium is bioengineered to include a cell surface protein antigen I/II, spaP gene.

5. The method of claim 4, wherein the bacterium produces hydrogen peroxide, thereby increasing the pH level.

6. The method of claim 5, wherein the bacterium capable of producing the biofilm is bioengineered to express a surface binding protein gene.

7. The method of claim 6, further comprising:
adhering the composition, comprising the bacterium capable of producing the biofilm and bioengineered to include the surface binding protein gene, to the one or more substrates at the oral cavity at least via the surface binding protein gene,
wherein the pH level, at the one or more substrates at the oral cavity, is increased above 5.5 after the composition is adhered to the one or more substrates at the oral cavity.

8. The method of claim 7, wherein the one or more substrates at the oral cavity comprises one or more teeth, wherein the surface binding protein gene of the bioengineered bacterium of the composition adheres at least to the one or more teeth at the oral cavity, and wherein the pH level, at the one or more teeth, is increased above 5.5 after the surface binding protein gene is adhered to the one or more teeth at the oral cavity.

9. The method of claim 8, wherein after adhering the composition, comprising the bacterium capable of producing the biofilm and bioengineered to include the surface binding protein gene, to the one or more teeth at the oral cavity at least via the surface binding protein gene, reducing or preventing the growth of a pathogenic bacteria at the one or more teeth at the oral cavity to which the composition is adhered.

10. The method of claim 9, wherein when the composition, comprising the bacterium capable of producing the biofilm and bioengineered to include the surface binding protein gene, is adhered to the one or more teeth at the oral cavity, the bacterium produces hydrogen peroxide at the one or more teeth to which it is adhered to thereby increase the pH level at the one or more teeth above 5.5.

11. The method of claim 10, wherein the bacterium is Lactobacillus reuteri.

12. The method of claim 11, wherein the bacterium is bioengineered to include a mucus-adhesion promoting protein.

13. The method of claim 12, wherein the composition further comprises at least one of: sucrose, glucose, maltose, or xylitol.

14. The method of claim 13, wherein the composition comprises the Lactobacillus reuteri bacterium and xylitol, and wherein the Lactobacillus reuteri bacterium is bioengineered to include a mucus-adhesion promoting protein.

15. The method of claim 1, wherein the composition is incorporated into a carrier that comprises one of: a toothpaste, a mouth rinse, a dental floss, a chewing gum, and a lozenge, and wherein the carrier is administered into the oral cavity to deliver the composition into the oral cavity of the subject.

16. The method of claim 15, wherein, in addition to the composition, the carrier further comprises an active ingredient selected from the group consisting of: sodium fluoride, stannous fluoride, sodium monofluorophosphate, and a combination thereof.

17. A bioengineered bacterium, comprising:
a bacterium from the genus Streptococcus containing therein cell surface protein antigen I/II, spaP and spxB genes capable of inducing production of a biofilm; and
wherein the bacterium is further capable of increasing a pH level above 5.5.

18. The bacterium of claim 17, wherein the bacterium is of the species Streptococcus oligofermentans, and wherein the cell surface protein antigen I/II, spaP and spxB genes are introduced via a DNA pFW5 vector.

19. The bacterium of claim 17, wherein the bacterium induces production of the biofilm that is adapted to adhere to a tooth tissue surface.

20. A bioengineered bacterium, comprising:
a bacterium from the genus Lactobacillus containing therein a mucus-adhesion promoting protein capable of inducing adherence of a biofilm containing the bacterium to a surface,
wherein the bacterium is of the species Lactobacillus reuteri, and wherein the mucus-adhesion promoting protein gene is introduced via pTRKH3-MapA.


SEQUENCE LISTING (3 sequences)

SEQ ID NO: 1 - DNA, 1731 bp, Streptococcus gordonii
  source 1..1731: mol_type = other DNA
atgactcaagggaaaattactgcatctgcagcaatgcttaacgtattgaaaacatggggcgtagacacaatctacggtatcccatcaggaacactcagctcactcatggacgctttagctgaagacaaagatatccgcttcttgcaagttcgccacgaagaaacaggtgctcttgcagcggtcatgcaagctaaatttggtggctcaatcggggttgctgttggttcaggtggtccaggtgcgactcacttgattaacggtgtttacgatgcagctatggataacactccattccttgctatccttggatcacgtccagttaacgaactcaacatggatgccttccaagaattgaaccaaaacccaatgtacaacggtatcgctgtttacaataaacgtgtcgcttacgctgagcaattgccaaaagtaatcgacgaagcttgccgtgctgcagtttctaaaaaaggtccagctgttgttgaaattccagtaaacttcggtttccaagaaatcgacgaaaactcatactacggttcaggttcatacgaacgttcattcatcgctcctgccttgaacgaagttgaaatcgacaaagctgttgaaatcttgaacaatgctgaacgcccagttatctacgctggttacggtggtgttaaagcaggtgaagtgattactgaattgtcacgtaaaatcaaagcaccaatcatcacaactggtaaaaactttgaagcctttgaatggaattatgaaggtttgacaggttctgcttaccgtgttggttggaaaccagccaacgaagttgtctttgaagcagacacagttcttttccttggttcaaacttcccatttgctgaagtttacgaagccttcaagaacactgaaaaattcatccaagtcgatatcgacccttacaaacttggtaaacgtcatgcccttgacgcttcaatccttggtgacgcaggtcaagcagcgaaagctatccttgataaagtaaacccagttgaatctactccatggtggcgtgcaaacgttaagaacaaccaaaactggcgcgattacatgaacaaactcgaaggtaaaactgagggtgaattgcaattgtatcaagtttacaatgcaatcaacaaacatgctgatcaagacgctatctattcaatcgacgtaggtgacactactcaaacatctactcgtcaccttcacatgacacctaagaacatgtggcgtacatctccactctttgcgacaatgggtattgcccttcctggtggtatcgctgctaagaaagacaatcctgatcgccaagtatggaacatcatgggtgacggtgcattcaacatgtgctacccagacgtgatcacaaacgttcaatacgaccttccagttatcaacgttgtcttctcaaatggtaaatatgccttcatcaaggacaaatacgaagacacaaacaaacacttgtttggttgtgacttccctaatgctgactatgcgaaaatcgctgaagcgcaaggtgctgtaggatttacagttgaccgtatcgaagatatcgatgcagttgttgcagaagctgttaaattgaacaaagaaggtaaaactgttgtgatcgatgctcgcatctctcaacatcgtccacttccagtagaagtacttgaattggatccaaaacttcactcagaagaagcaatcaaagccttcaaggaaaaatacgaagcagaagaactcgtaccattccgc

SEQ ID NO: 2 - DNA, 3956 bp, Lactobacillus reuteri
  source 1..3956: mol_type = other DNA
aatattgtgcagaagtttcttgacgatcaatttaaataaattttaggaattgtattgacaacttattttttataagttatgattttgacatactaagttaggaggaacacgtcatgtttaacaaacaattaattaacgctactgtttgttgttgtggatcgcgtcttatgcgacccacagtttggcgtgcttacttttagttgagtaactgctaacgtaatcgggaataaatgcataaggcgcaaatttaagtgagaagctcttgagtttgcgccttttttgtattcgtttttgcattctaaaggagatagatattatgaaattcaacacccaattaattcatggcggtaatagtgaggatccaacaactggtgccgtttcaacaccaatctaccgttcatcaacattccaccaacacgttcttggcggtgaacctaaatgggaatattctcgaacaggaaatccaacacgtgtatctcttgaaaaattaatcgcagagcttgaacatgggacagtcggctttgcatttgcttctggatctgctgctattcatgctactttttctctattctcgcaaggcgatcattttgtagttggtagtgatgtatatgggggaacgttccggttaatcaataaggtattaaaacgctttggctttgaatttactgttgttgatatgcaagaccttgaagcagtagaaaatgcaattcaagataataccgttgcagtttactttgaaacaccgactaatccgctcttacaaatcgctgatattaaagcaattgccgatatcgcaaagaagcatggagtaaagacaattgttgataatacctttgccactccttataatcaacaaccattaactctcggggcggatattgttgttcactccgcaaccaaatatttaggcggtcatagtgatgttgttgccggattagcagttactaacgatgatgaaattgctgaacaattagcattcctgcaaaactcaatcggtagtacgcttggtcctgacgatagttggctattacaacgggggatgaaaactctcggtgcccggatgcgagttcaccaagaaaatgcgaatgaagttattaacttcctccaaaatgatgaccacattggaaaaatttattatccaggcttaaaagatttccctggtcatgaggttgcagctagcaaatgcggaactttggagcaatgatctcctttgaacttaaggatagtttagatgcgagaagtttgttgaaagtctacaattgattgatcttgccgaaagtttggggggaattgaaagtttgattgaagttccagctgtcatgactcatggatctattccccgggaaatacgattagaaaacggaattaaagatgagttgattcgtctttcagtgggaattgaggaccctgaagatattattgctgacttgcaacaagcacttaaaaaattagattaattatagaagaagggaaactgtgacttatgtgggaaattatcaagacatctttaccacaactcttagcagcgggatttaaatatacgattccgttagcgattatttcatttttatttggactgattattgcactagtaacagctttaattcgtctctctcgtcaacgtggcgcattcatgattcttaaatggattgctagtttttatgtctggctttttcggtcaacaccgctgttggttcaattatttattgtattctttggactgccatatctgagaattaagggagtcctgccgcatggaataaaattagaaccatttacagccggcattattacattttcacttaataccggtgcttatgcttccgaaacaattcgggcagcgatcagttcagttccaactgggcaatgggaagcaggtgcagctatcggaatgacgcgactgcaaatcttgtggcgaattattttaccgcaagccttaagagtagctttaccacccttatcaaacagttttataagtttggtaaaggacacgtcattagctgcttcaattacaattatggaaatgtttgctgttagtcaacaaattgcggccgaaaattatcaaccattacttatgtatactttagtagcaatggtttacgcggcctttacgacaattttatcttatttccaaggatatcttgagcgggtagtagatcggcaagtgaatgcaaataataggtgagaaaaatgaaattaacaaatattaacaaaagttttggtaataaaactgtcttgaaaaataccacgttagggtttcctgcaggtaaaacaacagtaatggttggtccatctggatcgggtaaatcaacaattcttcgctcgcttaacttactagtaatgccagagagcggccagacgattttgatgatcatcatttggattttagtcaaaaagtaagcaataaagataagttggcaattcgtcaagaaacagggatggttttccaagactacaatcttttccctaataaaactgtccttggtaatattattgaaggaccaacacaggttttgaaacagccgaaaaaagaagcaattgcaaatgcgcataatttacttgctaaagtgggcttagcagattatggagaagcttatccgaatgaattatctggcggacaggcacagcgggtggcgattgcccgggcattggcaatgtcaccaaagtatatcttacttgatgaaccgacttctgcccttgatccagaattagagcttagtgtattgaaggttctgttgcaattagctgaagaaaaacaatcaatggtaattgttactcataatatggtctttgcgaaacatgttgcggataagattatttttgtcgagaacggtgaaattttatatgatggtactccagacgagttttttgctccagataatccaaacaaacgaattaagaactttatcgcctcgatgacaatggaaaatttaaaataaaggaagtttagaataatgaaattttggaagaaagcactattaacaattgtagccttaacggtcggtacccctgcaggaatcacaagtgtttctgctgcttcatcagctgttaattcagaattagttcataagggagaattaacaattggtcttgagggaacgtactctccgtactcttatcgtaaaaataacaaattaactggctttgaagtagatcttggtaaagcagttgctaaaaagatgggcttaaaagctaactttgtaccaactaaatgggattcgctaattgccggtcttggttcaggtaagtttgatgtagtaatgaacaacattacacagacacctgaacgggccaagcaatataatttctctaccccatatatcaagtcccggtttgcattaattgttcctactgatagtaacatcaaaagcttgaagaatattaaaggcaagaagattattgctggtacgggaactaataatgcgaatgtggtaaaaaaatataagggtaaccttacaccaaatggcgattttgctagttccttagatatgatcaagcaaggtcgggctgccgggacaattaactcccgtgaagcttggtacgcttacagcaagaagaacagtactaagggtctcaagatgattgatgtttctagtgaacaagatccagctaagatttcagcactttttaacaagaaagatactgctattcaatcttcctacaacaaggcacttaaggaacttcaacaagacggaacagtcaagaagctatctgaaaagtacttcggtgcagatattactgaataattaaaaaagatctctaacgtctgtgtgttacagtgttagagatcttttttgaaagctataaataaaatatatttatcacagcttttcacaaattatgttaaattattatgctaaagttcccattggatcccaaggcttaagaacgacggggctcttagcgtaatcacgtttaagatcttcgctaaggtacttcttattaataacgacttg

SEQ ID NO: 3 - DNA, 4861 bp, Streptococcus mutans
  source 1..4861: mol_type = other DNA
atttcagcaaaaattgacaaatcaaatcaattatattacaattttttaacgtatattacaaaaatatatttggaagatttattcagatttggaggatttatgaaagtcaaaaaaacttacggttttcgtaaaagtaaaattagtaaaacactgtgtggtgctgttctaggaacagtagcagcagtctctgtagcaggacaaaaggtttttgccgatgaaacgaccactactagtgatgtagatactaaagtagttggaacacaaactggaaatccagcgaccaatttgccagaggctcaaggaagtgcgagtaagcaagctgaacaaagtcaaaccaactggagagacaaatggttcataccattgaagtacctaaaactgatcttgatcaagcagcaaaagatgctaagtctgctggtgtcaatgttgtccaagatgccgatgttaataaaggaactgttaaaacagctgaagaagcagtccaaaaagaaactgaaattaaagaagattacacaaaacaagctgaggatattaagaagacaacagatcaatataaatcggatgtagctgctcatgaggcagaagttgctaaaatcaaagctaaaaatcaggcaactaaagaacagtatggaaaagatatggtagctcataaagccgaggttgaacgcattaatgctgcaaatgctgccagtaaaacagcttatgaagctaaattggctcaatatcaagcagatttagcagccgttcaaaaaaccaatgctgccaatcaagcatcctatcaaaaagcccttgctgcttatcaggctgaactgaaacgtgttcaggaagctaatgcagccgccaaagccgcttatgatactgctgtagcagcaaataatgccaaaaatacagaaattgccgctgccaatgaagaaattagaaaacgcaatgcaacggccaaagctgaatatgagactaagttagctcaatatcaagctgaactaaagcgtgttcaggaagctaatgccgcaaacgaagcagactatcaagctaaattgaccgcctatcaaacagagcttgctcgcgttcagaaagccaatgcagatgctaaagcggcctatgaagcagctgtagcagcaaataatgccaaaaatgcggcacttacagctgaaaatactgcaattaagcaacgcaatgagaatgctaaggcgacttatgaagctgcactcaagcaatatgaggctgatttggcagcggtgaaaaaagctaatgccgcaaacgaagcagactatcaagctaaattgaccgcctatcaaacagagctcgctcgcgttcaaaaggccaatgcggatgctaaagcggcctatgaagcagctggcagcaaataatgccgcaaatgcagcgctcacagctgaaaatactgcaattaagaagcgcaatgcggatgctaaagctgattacgaagcaaaacttgctaagtatcaagcagatcttgccaaatatcaaaaagatttagcagactatccagttaagttaaaggcatacgaagatgaacaagcttctattaaagctgcactggcagaacttgaaaaacataaaaatgaagacggaaacttaacagaaccatctgctcaaaatttggtctatgatcttgagccaaatgcgaacttatctttgacaacagatgggaagttccttaaggcttctgctgtggatgatgcttttagcaaaagcacttcaaaagcaaaatatgaccaaaaaattcttcaattagatgatctagatatcactaacttagaacaatctaatgatgttgcttcttctatggagctttatggcaattttggtgataaagctggctggtcaacgacagtaagcaataactcacaggttaaatggggatcggtacttttagagcgcggtcaaagcgcaacagctacatacactaacctgcagaattcttattacaatggtaaaaagatttctaaaattgtctacaagtatacagtggaccctaagtccaagtttcaaggtcaaaaggtttggttaggtatttttaccgatccaactttaggtgtttttgcttccgcttatacaggtcaagttgaaaaaaacacttctatttttattaaaaatgaattcactttctatgacgaagatggaaaaccaattaattttgataatgcccttctatcagtagcttctcttaaccgagaaaataattctattgagatggccaaagattatacgggtaaatttgtcaaaatctctggatcatctatcggtgaaaagaatggcatgatttatgctacagatactctcaactttaggcagggtcaaggtggtgctcgttggaccatgtataccagagctagcgaaccgggatctggctgggatagttcagatgcgcctaactcttggtatggtgctggtgctatccgcatgtctggtcctaataacagtgtgactttgggtgctatctcatcaacacttgttgtgcctgctgatcctacaatggcaattgaaaccggcaaaaaaccaaatatttggtattcattaaatggtaaaatccgtgcggttaatcttcctaaagttactaaggaaaaacccacacctccggttaaaccaacagctccaactaaaccaacttatgaaacagaaaagccattaaaaccggcaccagtagctccaaattatgaaaaggagccaacaccaccgacaagaacaccggatcaagcagagccaaagaaacccactccgccgacctatgaaacagaaaagccgttggagccagcacctgttgagccaagctatgaagcagagccaacaccgccgacaaggacaccggatcaggcagagccaaataaacccacaccgccgacctatgaaacagaaaagccgttggagccagcacctgttgagccaagctatgaagcagagccaacgccaccgacaccaacaccagatcaaccagaaccaaacaaacctgttgagccaacttatgaggttattccaacaccgccgactgatcctgtttatcaagatcttccaacacctccatctataccaactgttcatttccattactttaaactagctgttcagccgcaggttaacaaagaaattagaaacaataacgatgttaatattgacagaactttggtggctaaacaatctgttgttaagttccagctgaagacagcagatctccctgctggacgtgatgaaacaacttcctttgtcttggtagatcccctgccatctggttatcaatttaatcctgaagctacaaaagctgccagccctggctttgatgtcgcttatgataatgcaactaatacagtcaccttcaaggcaactgcagcaactttggctacgtttaatgctgatttgactaagtcagtggcaacgatttatccaacagtggtcggacaagttcttaatgatggcgcaacttataagaataatttctcgctcacagtcaatgatgcttatggcattaaatccaatgttgttcgggtgacaactcctggtaaaccaaatgatccagataacccaaataataattacattaagccaactaaggttaataaaaatgaaaatggcgttgttattgatggtaaaacagttcttgccggttcaacgaattattatgagctaacttgggatttggatcaatataaaaacgaccgctcttcagcagataccattcaacaaggattttactatgtagatgattatccagaagaagcgcttgaattgcgtcaggatttagtgaagattacagatgctaatggcaatgaagttactggtgttagtgtggataattatactagtcttgaagcagcccctcaagaaattagagatgttctttctaaggcaggaattagacctaaaggtgctttccaaattttccgtgccgataatccaagagaattttatgatacttatgtcaaaactggaattgatttgaagattgtatcaccaatggttgttaaaaaacaaatgggacaaacaggcgggagttatgaagatcaagcttaccaaattgactttggtaatggttatgcatcaaatatcgttatcaataatgttcctaagattaaccctaagaaagatgtgaccttaacacttgatccggctgatacaaataatgttgatggtcagactattccacttaatacagtctttaattaccgtttgattggtggcattatccctgcaaatcactcagaagaactctttgaatacaatttctatgatgattatgatcaaacaggagatcactatactggtcagtataaagtttttgccaaggttgatatcactcttaaaaacggtgttattatcaagtcaggtactgagttaactcagtatacgacagcggaagttgataccactaaaggtgctatcacaattaagttcaaggaagcctttctgcgttctgtttcaattgattcagccttccaagctgaaagttatatccaaatgaaacgtattgcggttggtacttttgaaaatacctatattaatactgtcaatggggtaacttacagttcaaatacagtgaaaacaactactcctgaggatcctgcagaccctactgatccgcaagatccatcatcaccgcggacttcaactgtaattatctacaaacctcaatcaactgcttatcaaccaagctctgtccaaaaaacgttaccaaatacgggagtaacaaacaatgcttatatgcctttacttggtattattggcttagttactagttttagtttgcttggcttaaaggctaagaagattgacagcatagatattacattagaattaaaaagtgagatgaagcgataaatcacagattgagcttttatctcattttttgatt